SEQUENCE LISTING (5 sequences)

SEQ ID NO: 1 - protein, 1873 aa, Homo sapiens
MEPSSPQDEGLRKKQPKKPVPEILPRPPRALFCLTLENPLRKACISIVEWKPFETIILLTIFANCVALAVYLPMPEDDNNSLNLGLEKLEYFFLIVFSIEAAMKIIAYGFLFHQDAYLRSGWNVLDFTIVFLGVFTVILEQVNVIQSHTAPMSSKGAGLDVKALRAFRVLRPLRLVSGVPSLQVVLNSIFKAMLPLFHIALLVLFMVIIYAIIGLELFKGKMHKTCYFIGTDIVATVENEEPSPCARTGSGRRCTINGSECRGGWPGPNHGITHFDNFGFSMLTVYQCITMEGWTDVLYWVNDAIGNEWPWIYFVTLILLGSFFILNLVLGVLSGEFTKEREKAKSRGTFQKLREKQQLDEDLRGYMSWITQGEVMDVEDFREGKLSLDEGGSDTESLYEIAGLNKIIQFIRHWRQWNRIFRWKCHDIVKSKVFYWLVILIVALNTLSIASEHHNQPLWLTRLQDIANRVLLSLFTTEMLMKMYGLGLRQYFMSIFNRFDCFVVCSGILEILLVESGAMTPLGISVLRCIRLLRIFKITKYWTSLSNLVASLLNSIRSIASLLLLLFLFIVIFALLGMQLFGGRYDFEDTEVRRSNFDNFPQALISVFQVLTGEDWTSMMYNGIMAYGGPSYPGMLVCIYFIILFVCGNYILLNVFLAIAVDNLAEAESLTSAQKAKAEEKKRRKMSKGLPDKSEEEKSTMAKKLEQKPKGEGIPTTAKLKIDEFESNVNEVKDPYPSADFPGDDEEDEPEIPLSPRPRPLAELQLKEKAVPIPEASSFFIFSPTNKIRVLCHRIVNATWFTNFILLFILLSSAALAAEDPIRADSMRNQILKHFDIGFTSVFTVEIVLKMTTYGAFLHKGSFCRNYFNMLDLLVVAVSLISMGLESSAISVVKILRVLRVLRPLRAINRAKGLKHVVQCMFVAISTIGNIVLVTTLLQFMFACIGVQLFKGKFFRCTDLSKMTEEECRGYYYVYKDGDPMQIELRHREWVHSDFHFDNVLSAMMSLFTVSTFEGWPQLLYKAIDSNAEDVGPIYNNRVEMAIFFIIYIILIAFFMMNIFVGFVIVTFQEQGETEYKNCELDKNQRQCVQYALKARPLRCYIPKNPYQYQVWYIVTSSYFEYLMFALIMLNTICLGMQHYNQSEQMNHISDILNVAFTIIFTLEMILKLMAFKARGYFGDPWNVFDFLIVIGSIIDVILSEIDTFLASSGGLYCLGGGCGNVDPDESARISSAFFRLFRVMRLIKLLSRAEGVRTLLWTFIKSFQALPYVALLIVMLFFIYAVIGMQMFGKIALVDGTQINRNNNFQTFPQAVLLLFRCATGEAWQEILLACSYGKLCDPESDYAPGEEYTCGTNFAYYYFISFYMLCAFLVINLFVAVIMDNFDYLTRDWSILGPHHLDEFKAIWAEYDPEAKGRIKHLDVVTLLRRIQPPLGFGKFCPHRVACKRLVGMNMPLNSDGTVTFNATLFALVRTALKIKTEGNFEQANEELRAIIKKIWKRTSMKLLDQVIPPIGDDEVTVGKFYATFLIQEHFRKFMKRQEEYYGYRPKKDIVQIQAGLRTIEEEAAPEICRTVSGDLAAEEELERAMVEAAMEEGIFRRTGGLFGQVDNFLERTNSLPPVMANQRPLQFAEIEMEEMESPVFLEDFPQDPRTNPLARANTNNANANVAYGNSNHSNSHVFSSVHYEREFPEETETPATRGRALGQPCRVLGPHSKPCVEMLKGLLTQRAMPRGQAPPAPCQCPRVESSMPEDRKSSTPGSLHEETPHSRSTRENTSRCSAPATALLIQKALVRGGLGTLAADANFIMATGQALADACQMEPEEVEIMATELLKGREAPEGMASSLGCLNLGSSLGSLDQHQGSQETLIPPRL

SEQ ID NO: 2 - protein, 2186 aa, Homo sapiens
MVNENTRMYIPEENHQGSNYGSPRPAHANMNANAAAGLAPEHIPTPGAALSWQAAIDAARQAKLMGSAGNATISTVSSTQRKRQQYGKPKKQGSTTATRPPRALLCLTLKNPIRRACISIVEWKPFEIIILLTIFANCVALAIYIPFPEDDSNATNSNLERVEYLFLIIFTVEAFLKVIAYGLLFHPNAYLRNGWNLLDFIIVVVGLFSAILEQATKADGANALGGKGAGFDVKALRAFRVLRPLRLVSGVPSLQVVLNSIIKAMVPLLHIALLVLFVIIIYAIIGLELFMGKMHKTCYNQEGIADVPAEDDPSPCALETGHGRQCQNGTVCKPGWDGPKHGITNFDNFAFAMLTVFQCITMEGWTDVLYWVNDAVGRDWPWIYFVTLIIIGSFFVLNLVLGVLSGEFSKEREKAKARGDFQKLREKQQLEEDLKGYLDWITQAEDIDPENEDEGMDEEKPRNMSMPTSETESVNTENVAGGDIEGENCGARLAHRISKSKFSRYWRRWNRFCRRKCRAAVKSNVFYWLVIFLVFLNTLTIASEHYNQPNWLTEVQDTANKALLALFTAEMLLKMYSLGLQAYFVSLFNRFDCFVVCGGILETILVETKIMSPLGISVLRCVRLLRIFKITRYWNSLSNLVASLLNSVRSIASLLLLLFLFIIIFSLLGMQLFGGKFNFDEMQTRRSTFDNFPQSLLTVFQILTGEDWNSVMYDGIMAYGGPSFPGMLVCIYFIILFICGNYILLNVFLAIAVDNLADAESLTSAQKEEEEEKERKKLARTASPEKKQELVEKPAVGESKEEKIELKSITADGESPPATKINMDDLQPNENEDKSPYPNPETTGEEDEEEPEMPVGPRPRPLSELHLKEKAVPMPEASAFFIFSSNNRFRLQCHRIVNDTIFTNLILFFILLSSISLAAEDPVQHTSFRNHILFYFDIVFTTIFTIEIALKILGNADYVFTSIFTLEIILKMTAYGAFLHKGSFCRNYFNILDLLVVSVSLISFGIQSSAINVVKILRVLRVLRPLRAINRAKGLKHVVQCVFVAIRTIGNIVIVTTLLQFMFACIGVQLFKGKLYTCSDSSKQTEAECKGNYITYKDGEVDHPIIQPRSWENSKFDFDNVLAAMMALFTVSTFEGWPELLYRSIDSHTEDKGPIYNYRVEISIFFIIYIIIIAFFMMNIFVGFVIVTFQEQGEQEYKNCELDKNQRQCVEYALKARPLRRYIPKNQHQYKVWYVVNSTYFEYLMFVLILLNTICLAMQHYGQSCLFKIAMNILNMLFTGLFTVEMILKLIAFKPKGYFSDPWNVFDFLIVIGSIIDVILSETNHYFCDAWNTFDALIVVGSIVDIAITEVNPAEHTQCSPSMNAEENSRISITFFRLFRVMRLVKLLSRGEGIRTLLWTFIKSFQALPYVALLIVMLFFIYAVIGMQVFGKIALNDTTEINRNNNFQTFPQAVLLLFRCATGEAWQDIMLACMPGKKCAPESEPSNSTEGETPCGSSFAVFYFISFYMLCAFLIINLFVAVIMDNFDYLTRDWSILGPHHLDEFKRIWAEYDPEAKGRIKHLDVVTLLRRIQPPLGFGKLCPHRVACKRLVSMNMPLNSDGTVMFNATLFALVRTALRIKTEGNLEQANEELRAIIKKIWKRTSMKLLDQVVPPAGDDEVTVGKFYATFLIQEYFRKFKKRKEQGLVGKPSQRNALSLQAGLRTLHDIGPEIRRAISGDLTAEEELDKAMKEAVSAASEDDIFRRAGGLFGNHVSYYQSDGRSAFPQTFTTQRPLHINKAGSSQGDTESPSHEKLVDSTFTPSSYSSTGSNANINNANNTALGRLPRPAGYPSTVSTVEGHGPPLSPAIRVQEVAWKLSSNRCHSRESQAAMAGQEETSQDETYEVKMNHDTEACSEPSLLSTEMLSYQDDENRQLTLPEEDKRDIRQSPKRGFLRSASLGRRASFHLECLKRQKDRGGDISQKTVLPLHLVHHQALAVAGLSPLLQRSHSPASFPRPFATPPATPGSRGWPPQPVPTLRLEGVESSEKLNSSFPSIHCGSWAETTPGGGGSSAARRVRPVSLMVPSQAGAPGRQFHGSASSLVEAVLISEGLGQFAQDPKFIEVTTQELADACDMTIEEMESAADNILSGGAPQSPNGALLPFVNCRDAGQDRAGGEEDAGCVRARGRPSEEELQDSRVYVSSL

SEQ ID NO: 3 - protein, 2181 aa, Homo sapiens
MMMMMMMKKMQHQRQQQADHANEANYARGTRLPLSGEGPTSQPNSSKQTVLSWQAAIDAARQAKAAQTMSTSAPPPVGSLSQRKRQQYAKSKKQGNSSNSRPARALFCLSLNNPIRRACISIVEWKPFDIFILLAIFANCVALAIYIPFPEDDSNSTNHNLEKVEYAFLIIFTVETFLKIIAYGLLLHPNAYVRNGWNLLDFVIVIVGLFSVILEQLTKETEGGNHSSGKSGGFDVKALRAFRVLRPLRLVSGVPSLQVVLNSIIKAMVPLLHIALLVLFVIIIYAIIGLELFIGKMHKTCFFADSDIVAEEDPAPCAFSGNGRQCTANGTECRSGWVGPNGGITNFDNFAFAMLTVFQCITMEGWTDVLYWVNDAIGWEWPWVYFVSLIILGSFFVLNLVLGVLSGEFSKEREKAKARGDFQKLREKQQLEEDLKGYLDWITQAEDIDPENEEEGGEEGKRNTSMPTSETESVNTENVSGEGENRGCCGSLWCWWRRRGAAKAGPSGCRRWGQAISKSKLSRRWRRWNRFNRRRCRAAVKSVTFYWLVIVLVFLNTLTISSEHYNQPDWLTQIQDIANKVLLALFTCEMLVKMYSLGLQAYFVSLFNRFDCFVVCGGITETILVELEIMSPLGISVFRCVRLLRIFKVTRHWTSLSNLVASLLNSMKSIASLLLLLFLFIIIFSLLGMQLFGGKFNFDETQTKRSTFDNFPQALLTVFQILTGEDWNAVMYDGIMAYGGPSSSGMIVCIYFIILFICGNYILLNVFLAIAVDNLADAESLNTAQKEEAEEKERKKIARKESLENKKNNKPEVNQIANSDNKVTIDDYREEDEDKDPYPPCDVPVGEEEEEEEEDEPEVPAGPRPRRISELNMKEKIAPIPEGSAFFILSKTNPIRVGCHKLINHHIFTNLILVFIMLSSAALAAEDPIRSHSFRNTILGYFDYAFTAIFTVEILLKMTTFGAFLHKGAFCRNYFNLLDMLVVGVSLVSFGIQSSAISVVKILRVLRVLRPLRAINRAKGLKHVVQCVFVAIRTIGNIMIVTTLLQFMFACIGVQLFKGKFYRCTDEAKSNPEECRGLFILYKDGDVDSPVVRERIWQNSDFNFDNVLSAMMALFTVSTFEGWPALLYKAIDSNGENIGPIYNHRVEISIFFIIYIIIVAFFMMNIFVGFVIVTFQEQGEKEYKNCELDKNQRQCVEYALKARPLRRYIPKNPYQYKFWYVVNSSPFEYMMFVLIMLNTLCLAMQHYEQSKMFNDAMDILNMVFTGVFTVEMVLKVIAFKPKGYFSDAWNTFDSLIVIGSIIDVALSEADPTESENVPVPTATPGNSEESNRISITFFRLFRVMRLVKLLSRGEGIRTLLWTFIKSFQALPYVALLIAMLFFIYAVIGMQMFGKVAMRDNNQINRNNNFQTFPQAVLLLFRCATGEAWQEIMLACLPGKLCDPESDYNPGEEYTCGSNFAIVYFISFYMLCAFLIINLFVAVIMDNFDYLTRDWSILGPHHLDEFKRIWSEYDPEAKGRIKHLDVVTLLRRIQPPLGFGKLCPHRVACKRLVAMNMPLNSDGTVMFNATLFALVRTALKIKTEGNLEQANEELRAVIKKIWKKTSMKLLDQVVPPAGDDEVTVGKFYATFLIQDYFRKFKKRKEQGLVGKYPAKNTTIALQAGLRTLHDIGPEIRRAISCDLQDDEPEETKREEEDDVFKRNGALLGNHVNHVNSDRRDSLQQTNTTHRPLHVQRPSIPPASDTEKPLFPPAGNSVCHNHHNHNSIGKQVPTSTNANLNNANMSKAAHGKRPSIGNLEHVSENGHHSSHKHDREPQRRSSVKRTRYYETYIRSDSGDEQLPTICREDPEIHGYFRDPHCLGEQEYFSSEECYEDDSSPTWSRQNYGYYSRYPGRNIDSERPRGYHHPQGFLEDDDSPVCYDSRRSPRRRLLPPTPASHRRSSFNFECLRRQSSQEEVPSSPIFPHRTALPLHLMQQQIMAVAGLDSSKAQKYSPSHSTRSWATPPATPPYRDWTPCYTPLIQVEQSEALDQVNGSLPSLHRSSWYTDEPDISYRTFTPASLTVPSSFRNKNSDKQRSADSLVEAVLISEGLGRYARDPKFVSATKHEIADACDLTIDEMESAASTLLNGNVRPRANGDVGPLSHRQDYELQDFGPGYSDEEPDPGRDEEDLADEMICITTL

SEQ ID NO: 4 - protein, 1977 aa, Homo sapiens
MSESEGGKDTTPEPSPANGAGPGPEWGLCPGPPAVEGESSGASGLGTPKRRNQHSKHKTVAVASAQRSPRALFCLTLANPLRRSCISIVEWKPFDILILLTIFANCVALGVYIPFPEDDSNTANHNLEQVEYVFLVIFTVETVLKIVAYGLVLHPSAYIRNGWNLLDFIIVVVGLFSVLLEQGPGRPGDAPHTGGKPGGFDVKALRAFRVLRPLRLVSGVPSLHIVLNSIMKALVPLLHIALLVLFVIIIYAIIGLELFLGRMHKTCYFLGSDMEAEEDPSPCASSGSGRACTLNQTECRGRWPGPNGGITNFDNFFFAMLTVFQCVTMEGWTDVLYWMQDAMGYELPWVYFVSLVIFGSFFVLNLVLGVLSGEFSKEREKAKARGDFQKQREKQQMEEDLRGYLDWITQAEELDMEDPSADDNLGSMAEEGRAGHRPQLAELTNRRRGRLRWFSHSTRSTHSTSSHASLPASDTGSMTETQGDEDEEEGALASCTRCLNKIMKTRVCRRLRRANRVLRARCRRAVKSNACYWAVLLLVFLNTLTIASEHHGQPVWLTQIQEYANKVLLCLFTVEMLLKLYGLGPSAYVSSFFNRFDCFVVCGGILETTLVEVGAMQPLGISVLRCVRLLRIFKVTRHWASLSNLVASLLNSMKSIASLLLLLFLFIIIFSLLGMQLFGGKFNFDQTHTKRSTFDTFPQALLTVFQILTGEDWNVVMYDGIMAYGGPFFPGMLVCIYFIILFICGNYILLNVFLAIAVDNLASGDAGTAKDKGGEKSNEKDLPQENEGLVPGVEKEEEEGARREGADMEEEEEEEEEEEEEEEEEGAGGVELLQEVVPKEKVVPIPEGSAFFCLSQTNPLRKGCHTLIHHHVFTNLILVFIILSSVSLAAEDPIRAHSFRNHILGYFDYAFTSIFTVEILLKMTVFGAFLHRGSFCRSWFNMLDLLVVSVSLISFGIHSSAISVVKILRVLRVLRPLRAINRAKGLKHVVQCVFVAIRTIGNIMIVTTLLQFMFACIGVQLFKGKFYTCTDEAKHTPQECKGSFLVYPDGDVSRPLVRERLWVNSDFNFDNVLSAMMALFTVSTFEGWPALLYKAIDAYAEDHGPIYNYRVEISVFFIVYIIIIAFFMMNIFVGFVIITFRAQGEQEYQNCELDKNQRQCVEYALKAQPLRRYIPKNPHQYRVWATVNSAAFEYLMFLLILLNTVALAMQHYEQTAPFNYAMDILNMVFTGLFTIEMVLKIIAFKPKHYFTDAWNTFDALIVVGSIVDIAVTEVNNGGHLGESSEDSSRISITFFRLFRVMRLVKLLSKGEGIRTLLWTFIKSFQALPYVALLIAMIFFIYAVIGMQMFGKVALQDGTQINRNNNFQTFPQAVLLLFRCATGEAWQEIMLASLPGNRCDPESDFGPGEEFTCGSNFAIAYFISFFMLCAFLIINLFVAVIMDNFDYLTRDWSILGPHHLDEFKRIWSEYDPGAKGRIKHLDVVALLRRIQPPLGFGKLCPHRVACKRLVAMNMPLNSDGTVTFNATLFALVRTSLKIKTEGNLEQANQELRIVIKKIWKRMKQKLLDEVIPPPDEEEVTVGKFYATFLIQDYFRKFRRRKEKGLLGNDAAPSTSSALQAGLRSLQDLGPEMRQALTCDTEEEEEEGQEGVEEEDEKDLETNKATMVSQPSARRGSGISVSLPVGDRLPDSLSFGPSDDDRGTPTSSQPSVPQAGSNTHRRGSGALIFTIPEEGNSQPKGTKGQNKQDEDEEVPDRLSYLDEQAGTPPCSVLLPPHRAQRYMDGHLVPRRRLLPPTPAGRKPSFTIQCLQRQGSCEDLPIPGTYHRGRNSGPNRAQGSWATPPQRGRLLYAPLLLVEEGAAGEGYLGRSSGPLRTFTCLHVPGTHSDPSHGKRGSADSLVEAVLISEGLGLFARDPRFVALAKQEIADACRLTLDEMDNAASDLLAQGTSSLYSDEESILSRFDEEDLGDEMACVHAL

SEQ ID NO: 5 - protein, 1977 aa, Homo sapiens
MSESEGGKDTTPEPSPANGAGPGPEWGLCPGPPAVEGESSGASGLGTPKRRNQHSKHKTVAVASAQRSPRALFCLTLANPLRRSCISIVEWKPFDILILLTIFANCVALGVYIPFPEDDSNTANHNLEQVEYVFLVIFTVETVLKIVAYGLVLHPSAYIRNGWNLLDFIIVVVGLFSVLLEQGPGRPGDAPHTGGKPGGFDVKALRAFRVLRPLRLVSGVPSLHIVLNSIMKALVPLLHIALLVLFVIIIYAIIGLELFLGRMHKTCYFLGSDMEAEEDPSPCASSGSGRACTLNQTECRGRWPGPNGGITNFDNFFFAMLTVFQCVTMEGWTDVLYWMQDAMGYELPWVYFVSLVIFGSFFVLNLVLGVLSGEFSKEREKAKARGDFQKQREKQQMEEDLRGYLDWITQAEELDMEDPSADDNLGSMAEEGRAGHRPQLAELTNRRRGRLRWFSHSTRSTHSTSSHASLPASDTGSMTETQGDEDEEEGALASCTRCLNKIMKTRVCRRLRRANRVLRARCRRAVKSNACYWAVLLLVFLNTLTIASEHHGQPVWLTQIQEYANKVLLCLFTVEMLLKLYGLGPSAYVSSFFNRFDCFVVCGGILETTLVEVGAMQPLGISVLRCVRLLRIFKVTRHWASLSNLVASLLNSMKSIASLLLLLFLFIIIFSLLGMQLFGGKFNFDQTHTKRSTFDTFPQALLTVFQILTGEDWNVVMYDGIMAYGGPFFPGMLVCIYFIILFICGNYILLNVFLATAVDNLASGDAGTAKDKGGEKSNEKDLPQENEGLVPGVEKEEEEGARREGADMEEEEEEEEEEEEEEEEEGAGGVELLQEVVPKEKVVPIPEGSAFFCLSQTNPLRKGCHTLIHHHVFTNLILVFIILSSVSLAAEDPIRAHSFRNHILGYFDYAFTSIFTVEILLKMTVFGAFLHRGSFCRSWFNMLDLLVVSVSLISFGIHSSAISVVKILRVLRVLRPLRAINRAKGLKHVVQCVFVAIRTIGNIMIVTTLLQFMFACIGVQLFKGKFYTCTDEAKHTPQECKGSFLVYPDGDVSRPLVRERLWVNSDFNFDNVLSAMMALFTVSTFEGWPALLYKAIDAYAEDHGPIYNYRVEISVFFIVYIIIIAFFMMNIFVGFVIITFRAQGEQEYQNCELDKNQRQCVEYALKAQPLRRYIPKNPHQYRVWATVNSAAFEYLMFLLILLNTVALAMQHYEQTAPFNYAMDILNMVFTGLFTIEMVLKIIAFKPKHYFTDAWNTFDALIVVGSIVDIAVTEVNNGGHLGESSEDSSRISITFFRLFRVMRLVKLLSKGEGIRTLLWTFIKSFQALPYVALLIAMIFFIYAVIGMQMFGKVALQDGTQINRNNNFQTFPQAVLLLFRCATGEAWQEIMLASLPGNRCDPESDFGPGEEFTCGSNFAIAYFISFFMLCAFLIINLFVAVIMDNFDYLTRDWSILGPHHLDEFKRIWSEYDPGAKGRIKHLDVVALLRRIQPPLGFGKLCPHRVACKRLVAMNMPLNSDGTVTFNATLFALVRTSLKIKTEGNLEQANQELRIVIKKIWKRMKQKLLDEVIPPPDEEEVTVGKFYATFLIQDYFRKFRRRKEKGLLGNDAAPSTSSALQAGLRSLQDLGPEMRQALTCDTEEEEEEGQEGVEEEDEKDLETNKATMVSQPSARRGSGISVSLPVGDRLPDSLSFGPSDDDRGTPTSSQPSVPQAGSNTHRRGSGALIFTIPEEGNSQPKGTKGQNKQDEDEEVPDRLSYLDEQAGTPPCSVLLPPHRAQRYMDGHLVPRRRLLPPTPAGRKPSFTIQCLQRQGSCEDLPIPGTYHRGRNSGPNRAQGSWATPPQRGRLLYAPLLLVEEGAAGEGYLGRSSGPLRTFTCLHVPGTHSDPSHGKRGSADSLVEAVLISEGLGLFARDPRFVALAKQEIADACRLTLDEMDNAASDLLAQGTSSLYSDEESILSRFDEEDLGDEMACVHAL

(19) United States
(12) Patent Application Publication
(10) Pub. No.: US 2025/0222028 A1
(43) Pub. Date: Jul. 10, 2025

(54) ENGINEERED IMMUNE CELL PLATFORM

(71) Applicant: Cancer Research Technology Limited, London (GB)

(72) Inventor: Maike De La Roche, Cambridge (GB)

(73) Assignee: Cancer Research Technology Limited, London (GB)

(21) Appl. No.: 18/851,484

(22) PCT Filed: Mar. 28, 2023
(86) PCT No.: PCT/EP2023/058052

§ 371 Date: Sep. 26, 2024

(30) Foreign Application Priority Data
Mar. 28, 2022 (GB) 2204386.3

(51) Int. Cl. A61K 35/17; A61K 40/11; A61K 40/42; A61P 35/00; C07K 14/705; C12N 5/0783

(52) U.S. Cl. CPC A61K 35/17; A61K 40/11; A61K 40/42; A61P 35/00; C07K 14/705; C12N 5/0636; C12N 5/0646; A61K 2239/59; C12N 2501/999; C12N 2510/00

(57) ABSTRACT

The invention provides an engineered immune cell, wherein the engineered immune cell comprises a gain-of-function mutation in a Cav channel and/or overexpresses an L-type voltage-gated calcium (Cav) channel. Also provided are related engineered immune cells for use in medicine. Also provided are L-type Cav channel agonists for use in a method of stimulating cell killing activity of an immune cell in a subject having a proliferative disorder. Pharmaceutical compositions and methods are also provided.

### FIELD OF THE INVENTION

The present invention relates to products and methods for modulating, including enhancing, immune cell cytotoxicity. In particular, enhancement of L-type Cav channel expression or activity is disclosed for use in medicine. The invention provides engineered immune cells, agents and methods for engineering the immune cells, as well as medical uses thereof.

### BACKGROUND

Cytotoxic T lymphocytes (CTLs) play a critical role in the immune response against cancer and intracellular pathogens. CTLs recognise tumour and infected cells via their T cell receptor complex (TCR) and this recognition induces a cascade of intracellular signaling pathways. One such pathway is the Hedgehog (Hh) signaling pathway. Induction of Hh leads to enhanced expression of the Hh transcription factor Gli1 (de la Roche, 2013). GLI1 expression is therefore a faithful reporter of Hh signaling. The inventors have previously shown that Gli1 induction is Ca²⁺-dependent (Hanna, 2021) and is required for CD8⁺ T cell killing (Hanna, 2021 & de la Roche, 2013). They have further shown that this non-canonical Hh signaling pathway acts through L-type voltage-gated Ca²⁺ channels.

There are four members in the L-type voltage-gated calcium channel family. These are Cav1.1, Cav1.2, Cav1.3 and Cav1.4. All four members are expressed in murine T cells (Badou, 2013), whereas only Cav1.4 and, to a lesser extent, Cav1.3 are expressed in human CD8+ T cells (Hanna, 2021).

Cytotoxic lymphocytes include CD8⁺ T cells, CD4⁺ T cells and natural killer (NK) cells, which all exert their effects on tumour and viral-infected cells by recognising different targets. For example, CD8⁺ T cells recognise major histocompatibility complex class I (MHCI) antigens, whereas CD4⁺ T cells recognise MHC class II antigens. In contrast, NK cells recognise and kill MHC-negative cells.

Immune cell therapies are among the most advanced in the class of cell therapies, having already demonstrated definitive evidence of clinical benefits in cancer and infectious disease. The types of immune cells that can be used as therapeutic agent include alpha beta T cells, NK cells, gammadelta (gd) T Cells, NK T (NKT) cells, and Induced Pluripotent Stem Cell (iPSC)-Derived Immune Effectors. The applicability of immune cell therapy is broad and includes the treatment of cancer, infection, allogeneic transplantation and autoimmunity.

T cell therapies emerge as the most advanced within this therapeutic class. To treat cancer, T cell immunotherapy can be deployed in different ways: Non-engineered Tumour infiltrating lymphocytes (TILs), T cells Expressing Engineered T Cell Receptors (TCRs), Chimeric Antigen Receptor (CAR) T cells.

NK cells application in cancer treatment is under investigation as well. The main advantage of NK cells is that they possess cytotoxic capacity, but do not mediate severe graft versus host disease (GVHD) when administered to the patient as allogenic transplant, potentially enabling the development of an “off-the-shelf” product.

There are currently 4 adoptive cell therapies that are approved by the FDA for the treatment of lymphoma, leukemia and myeloma subtypes. Despite these advances, there remains a need for methods and agents for improving the effectiveness of cell therapies, e.g., to treat conditions such as cancer.

The present invention has been devised in light of the above considerations.

### SUMMARY OF THE INVENTION

The inventors sought to provide a platform that enhances the cytotoxicity of immune cells, by increasing the expression or activity of L-type Cav channels. This may find applications in adoptive cell therapy.

Accordingly, in a first aspect, the invention provides an engineered immune cell that overexpresses an L-type voltage-gated calcium (Cav) channel and/or comprises a gain-of-function mutation (GOF) in an L-type Cav channel.

In some embodiments, the engineered immune cell overexpresses Cav1.4 in comparison with a wild-type immune cell of the same type. In other embodiments, the engineered immune cell overexpresses Cav1.3 in comparison with a wild-type immune cell of the same type. In other embodiments, the engineered immune cell overexpresses Cav1.2 in comparison with a wild-type immune cell of the same type. In other embodiments, the engineered immune cell overexpresses Cav1.1 in comparison with a wild-type immune cell of the same type.

In some embodiments, the engineered immune cell comprises a mutation in the gene encoding the L-type Cav channel. In some embodiments, the engineered immune cell comprises a gain-of-function mutation in CACNA1F (the gene that encodes Cav1.4). In some embodiments, the CACNA1F mutation comprises I745T (using EU numbering) (Hemara-Wahanui, 2005). This mutation is set out in SEQ ID NO: 5. This mutation is also referred to in the literature as I756T (Williams, 2020). The protein structures of the pore-forming Ca²⁺-channel α1-subunits are highly conserved, and thus the same I745T GOF mutation in CACNA1F can be predicted to cause a gain-of-function in the other L-type Cav channels, since the region is highly conserved.

Other gain-of-function mutations that are encompassed by the present invention are set out in Table 1 below.

TABLE 1
Overview of gain of function (GOF) mutations in L-type Cav
channels Cav1.4, Cav1.3 and Cav1.2 (Striessnig, 2021)

| Mutation | Channel | Comments |
|---|---|---|
| G369D | Cav1.4 | Patient mutations also found in Cav1.2 and Cav1.3 |
| F753C | Cav1.4 | Patient mutations also found in Cav1.3 |
| I745T | Cav1.4 | Patient mutations also found in Cav1.3 |
| (I756T) | | |
| G403D | Cav1.3 | Patient mutations found in Cav1.3 and Cav1.2 |
| G407R | Cav1.3 | Less severe phenotype, patient mutations also found |
| | | in Cav1.2 |
| V401L | Cav1.3 | — |
| A749T | Cav1.3 | — |
| A749G | Cav1.3 | Less severe phenotype |
| I750M | Cav1.3 | Patient mutations also found in Cav1.4 |
| G402R/S | Cav1.2 | Patient mutations also found in Cav1.3 and Cav1.4 |
| E407A | Cav1.2 | — |
| I1186T | Cav1.2 | — |

As the L-type Cav channels in T cells may not be voltage-gated (Kotturi, 2005) preferred mutations are not within the voltage sensor region of the L-type Cav channels. Preferred mutations are located within the activation gate, i.e., the cytoplasmic region of the S6 helix; within the S5-S6 linker (Striessnig, 2021); or within the S5-S6 pore. GOF mutations that lead to conversion of the Ca²⁺ channel in a non-selective cation channel are not preferable, because Ca²⁺ transport is essential for induction of Gli1 (Hanna, 2021).

In other aspects, the invention provides an engineered immune cell which comprises a mutation in a gene encoding a component of the Hh signaling pathway, which leads to an increase in GLI1 expression and/or activity, which can be measured by a Gli1 reporter assay. For example, the mutation may be in the PTCH1 gene, which encodes the protein patched homolog 1 (PTCH1). PTCH1 enhances canonical Hh signalling.

In a related aspect, the invention provides an engineered immune cell which comprises a mutation in a gene encoding a component of the Hh signaling pathway, which leads to an increase in GLI1 expression and/or activity, for use in medicine (e.g. for use in the treatment of a proliferative disorder in a mammal). In some embodiments the mutation may be in the PTCH1 gene, which encodes the protein patched homolog 1 (PTCH1) (e.g. a PTCH1 mutation that has been found to be present in a subject having Gorlin Syndrome or a mutation that has been artificially introduced into PTCH1 in order to silence, down-regulate or otherwise reduce the ability of PTCH1 to act as a negative regulator of GLI1.

In some embodiments, the engineered immune cell is selected from the group consisting of a T cell (including a gamma delta (γδ) T cell), a chimeric antigen receptor (CAR)-T cell, an NK cell and a CAR-NK cell. The inventors have surprisingly found that calcium-dependent Gli1 signalling is conserved between T cells and NK cells. In some embodiments, the engineered immune cell is a T cell. In some embodiments, the T cell is a CD8⁺ T cell. In some embodiments, the T cell is a CD4⁺ T cell. In some embodiments, the T cell is a cytotoxic CD4⁺ T cell. In some embodiments, the T cell is a γδ T cell.

In a second aspect, the invention provides an engineered immune cell for use in medicine, wherein the engineered immune cell overexpresses an L-type Cav channel and/or comprises a gain-of-function mutation in an L-type Cav channel. In some embodiments, the engineered immune cell is for use in a method of treating chronic infection in a subject. For example, the engineered immune cell may be for use in a method of treating SARS-CoV-II, HIV, hepatitis C, hepatitis B, human cytomegalovirus (CMV) or opportunistic fungal infection such as aspergillosis. In some embodiments, the engineered immune cell is for use in a method of treating an autoimmune disease in a subject. For example, the engineered immune cell may be for use in a method of treating rheumatoid arthritis (RA), Type 1 diabetes, psoriasis, psoriatic arthritis, multiple sclerosis (MS), systemic lupus erythematosus (SLE), lupus nephritis, inflammatory bowel disease (IBD), autoimmune Addison's disease (AAD), Grave's disease, Sjögren's syndrome, Hashimoto's thyroiditis, Myasthenia gravis or Autoimmune vasculitis.

In a third aspect, the invention provides an engineered immune cell for use in a method of treating cancer in a subject, wherein the engineered immune cell overexpresses an L-type Cav channel and/or comprises a gain-of-function mutation in an L-type Cav channel. Particular target cancers of interest include those cancers with high infiltration of cytotoxic lymphocytes. This includes melanoma, lung cancer, colorectal cancer and breast cancer. Other cancers of interest are those that are sensitive to CAR T and NK cell therapy. These cancers include blood cancers, such as lymphoma, leukemia (for example ALL and CLL) and multiple myeloma; and solid tumours targeted by CARs, including lung cancer, liver cancer, stomach cancer (including those EGFR)/CNS, pediatric glioma (HER2)/ovarian, cervical, pancreatic, lung (Mesothelin), colon, pancreatic, prostate, gastric, liver (EpCAM), and many more.

In some embodiments, the engineered immune cell overexpresses Cav1.4 in comparison with a wild-type immune cell of the same type.

In some embodiments, the engineered immune cell comprises a gain-of-function mutation in CACNA1F.

In some embodiments, the CACNA1F mutation comprises I745T.

In some embodiments, the engineered immune cell is selected from the group consisting of: a T cell (including a gamma delta (γδ) T cell), a CAR-T cell, an NK cell and a CAR-NK cell.

In some embodiments, the method of treating cancer in a subject is an in vivo method. Such a method may include adoptive transfer of e.g., T cells, as described herein. In these embodiments, the method may comprise the removal of immune cells from a subject and subsequent manipulation. The manipulation may include genetic engineering to introduce a gain-of-function mutation in an L-type Cav channel, or to overexpress an L-type Cav channel. In some embodiments, the method may include the step of administering the manipulated immune cells to a subject. The subject may be the same subject or a different subject as from where the immune cells were removed, i.e. the method may be allogeneic or autologous. In some embodiments, the method of stimulating T cell killing activity is an ex vivo method, i.e., the method comprises a step of manipulating the immune cells outside of the body of the subject. In such a method, it is contemplated that the immune cells may be administered back to the subject after the ex vivo manipulation.

In a fourth aspect, the invention provides an L-type Cav channel agonist for use in a method of stimulating cell killing activity of an immune cell in a subject having a proliferative disorder. In some embodiments, the method of stimulating cell killing activity is an in vivo method. The immune cell may be a T cell (including a gamma delta (γδ) T cell), a CAR-T cell, an NK cell or a CAR-NK cell. Such a method may include adoptive transfer of e.g., T cells, as described herein. In these embodiments, the method may comprise the removal of immune cells from a subject and subsequent manipulation. The manipulation may include treatment with an L-type Cav channel agonist. In some embodiments, the method may include the step of administering the manipulated immune cells to a subject. The subject may be the same subject or a different subject as from where the immune cells were removed, i.e. the method may be allogeneic or autologous. In some embodiments, the method of stimulating T cell killing activity is an ex vivo method, i.e., the method comprises a step of manipulating the immune cells outside of the body of the subject. In such a method, it is contemplated that the immune cells may be administered back to the subject after the ex vivo manipulation. In some embodiments, the L-type Cav channel agonist for use in accordance with this aspect of the invention exhibits Gli1 enhancing activity in an immune cell, as determined by a Gli1 reporter assay. For example, a Gli1 reporter assay as defined herein.

In a fifth aspect, the invention provides a pharmaceutical composition comprising an immune cell that has been treated with, or which is in admixture with, an L-type Cav channel agonist. The immune cell may be a T cell (including a gamma delta (γδ) T cell), a CAR-T cell, an NK cell or a CAR-NK cell. In some embodiments, the composition is for use in a method of stimulating cell killing activity against a target cell population (e.g. a diseased cell population, such as a cancer cell population). The composition may be for use in an in vivo, ex vivo, or in vitro method.

In a sixth aspect, the invention provides an in vitro method of increasing cell killing activity of an immune cell, comprising bringing an L-type Cav channel agonist into contact with the immune cell, wherein the Cav channel agonist is capable of stimulating L-type Cav channel activity in a Gli1 reporter assay. In some embodiments the L-type Cav channel agonist is incubated with the immune cell for a period sufficient to bring about an increase in cell killing activity and/or cell killing potential. For example, the incubation may be for 10, 20, 30 minutes, or 1, 2, 5, 12, 24, 48 hours or more.

In some embodiments, in accordance with any aspect of the present invention, the L-type Cav channel agonist is a compound as disclosed in EP 0230110 A1 and EP 0300688 A1, the contents of which are incorporated herein by reference. For example, the L-type Cav channel agonist may be a compound as defined by the formula in claim 1 of EP 0230110 A1. For example, the L-type Cav channel agonist may be a compound as defined by the formula in claim 1 of EP 0300688 A1. In some embodiments, the L-type Cav channel agonist is selected from the group consisting of FPL 64176 and Bay K8644.

In a seventh aspect, the invention provides an in vitro method of engineering an immune cell to increase cell killing activity, comprising genetically engineering an immune cell to enhance expression of or to stimulate L-type Cav channel activity in a Gli1 reporter assay.

In some embodiments, said engineering comprises overexpressing Cav1.4. In some embodiments, the method of overexpression comprises the use of a vector to stably express Cav1.4. In some embodiments, the vector is a lentiviral vector, a retroviral vector, or a transposon-based vector.

In some embodiments, said engineering comprises mutating Cav1.4 to introduce a gain-of-function mutation. In some embodiments, the Cav1.4 mutation comprises I745T.

In other embodiments, said engineering comprises nuclease-based editing. In some embodiments, the engineering comprises CRISPR-based editing. In some embodiments, the engineering comprises prime editing (Anzalone, 2019).

In some embodiments, the immune cell is selected from the group consisting of a T cell (including a gamma delta (γδ) T cell), a CAR-T cell, an NK cell and a CAR-NK cell.

In other related aspects, the invention provides a method of treatment of a proliferative disorder in a mammalian subject, comprising administering a therapeutically effective amount of an engineered immune cell to the subject in need thereof, wherein the immune cell overexpresses an L-type Cav channel and/or comprises a gain-of-function mutation in an L-type Cav channel.

In some embodiments, the immune cell is selected from the group consisting of a T cell (including a gamma delta (γδ) T cell), a CAR-T cell, an NK cell and a CAR-NK cell.

In other related aspects, the invention provides a method of treatment of a proliferative disorder in a mammalian subject, comprising administering a therapeutically effective amount of an L-type Cav channel agonist to the subject in need thereof.

In another aspect, the invention provides a method of treatment of a proliferative disorder in a mammalian subject, comprising administering a therapeutically effective amount of an immune cell treated with an L-type Cav channel agonist to the subject in need thereof. In some embodiments, the immune cell is derived from the subject in need thereof, i.e., it is an autologous method of treatment. In some embodiments, the immune cell is not derived from the subject in need thereof, i.e., it is an allogeneic method of treatment. In preferred embodiments, the immune cell is an NK cell.

In some embodiments, the L-type Cav channel agonist is selected from the group consisting of FPL 64176 and Bay K8644.

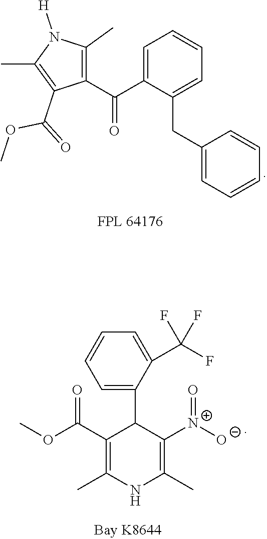 [SMILES: COC(=O)c1c(C)[nH]c(C)c1C(=O)c1ccccc1Cc1ccccc1 ; COC(=O)C1=C(C)NC(C)=C([N+](=O)[O-])C1c1ccccc1C(F)(F)F]

In other aspects, the invention provides a method for determining the cytotoxic fitness of an immune cell from a mammalian subject, comprising measuring the level of GLI1 expression in the immune cell from the mammalian subject. The method may be carried out in vitro or in vivo. In particular, GLI1 expression may be measured using a Gli1 reporter assay as defined herein. By cytotoxic fitness, it is meant the level of cytotoxicity of an immune cell. In this way, GLI1 expression is provided as a biomarker for the cytotoxic ability of the immune system of the subject or of an immune cell.

In some embodiments, the immune cell is an engineered immune cell. In some embodiments, the method comprises administering a test agent to the immune cell. In some embodiments, the test agent is an L-type Cav channel agonist. In some embodiments, the engineered immune cell is a CAR T cell, a CAR NK cell, a T cell (including a gamma delta (γδ) T cell), a or an NK cell. Therefore, the invention provides GLI1 as a biomarker for determining the cytotoxic effectiveness of a cell for use in adoptive cell therapy.

In another aspect, the present invention provides an engineered immune cell for use in a method of treating a proliferative disorder in a subject, wherein the engineered immune cell comprises a loss-of-function mutation in PTCH1 and/or wherein PTCH1 has been silenced or down-regulated and/or wherein the PTCH1 gene product has been antagonised. In some embodiments the loss-of-function mutation in PTCH1 is a mutation that has been found to be present in a human patient having Gorlin Syndrome. In some embodiments the engineered immune cell for use is selected from the group consisting of a T cell (including a gamma delta (γδ) T cell), a CAR-T cell, an NK cell, and a CAR-NK cell. In some embodiments the proliferative disorder comprises a cancer, optionally wherein the cancer is selected from the group consisting of: melanoma, lung cancer, colorectal cancer, breast cancer, lymphoma, leukaemia, multiple myeloma, liver cancer, stomach cancer, central nervous system (CNS) tumour, paediatric glioma, ovarian cancer, cervical cancer, colon cancer, pancreatic cancer, prostate cancer, and gastric cancer.

In another aspect, the present invention provides a method of treatment of a proliferative disorder in a mammalian subject, comprising administering a therapeutically effective amount of an engineered immune cell to the subject in need thereof, wherein the engineered immune cell comprises a loss-of-function mutation in PTCH1 and/or wherein PTCH1 has been silenced or down-regulated and/or wherein the PTCH1 gene product has been antagonised. In some embodiments the loss-of-function mutation in PTCH1 is a mutation that has been found to be present in a human patient having Gorlin Syndrome. In some embodiments the engineered immune cell is selected from the group consisting of a T cell (including a gamma delta (γδ) T cell), a CAR-T cell, an NK cell, and a CAR-NK cell. In some embodiments the proliferative disorder comprises a cancer. For example a cancer selected from the group consisting of: melanoma, lung cancer, colorectal cancer, breast cancer, lymphoma, leukaemia, multiple myeloma, liver cancer, stomach cancer, central nervous system (CNS) tumour, paediatric glioma, ovarian cancer, cervical cancer, colon cancer, pancreatic cancer, prostate cancer, and gastric cancer.

The invention includes the combination of the aspects and preferred features described except where such a combination is clearly impermissible or expressly avoided.

### Sequences

Cav1.1 (CACNA1S)
The amino acid sequence of human Cav1.1 (encoded by CACNA1S) is set forth in SEQ ID NO: 1:
(SEQ ID NO: 1)
MEPSSPQDEGLRKKQPKKPVPEILPRPPRALFCLTLENPLRKACISIVEWKPFETIILLTIFANCVALAV
YLPMPEDDNNSLNLGLEKLEYFFLIVFSIEAAMKIIAYGFLFHQDAYLRSGWNVLDFTIVFLGVFTVILE
QVNVIQSHTAPMSSKGAGLDVKALRAFRVLRPLRLVSGVPSLQVVLNSIFKAMLPLFHIALLVLFMVIIY
AIIGLELFKGKMHKTCYFIGTDIVATVENEEPSPCARTGSGRRCTINGSECRGGWPGPNHGITHFDNFGF
SMLTVYQCITMEGWTDVLYWVNDAIGNEWPWIYFVTLILLGSFFILNLVLGVLSGEFTKEREKAKSRGTEF
QKLREKQQLDEDLRGYMSWITQGEVMDVEDFREGKLSLDEGGSDTESLYEIAGLNKIIQFIRHWRQWNRI
FRWKCHDIVKSKVFYWLVILIVALNTLSIASEHHNQPLWLTRLQDIANRVLLSLFTTEMLMKMYGLGLRQ
YFMSIFNRFDCFVVCSGILEILLVESGAMTPLGISVLRCIRLLRIFKITKYWTSLSNLVASLLNSIRSIA
SLLLLLFLFIVIFALLGMQLFGGRYDFEDTEVRRSNFDNFPQALISVFQVLTGEDWTSMMYNGIMAYGGP
SYPGMLVCIYFIILFVCGNYILLNVFLAIAVDNLAEAESLTSAQKAKAEEKKRRKMSKGLPDKSEEEKST
MAKKLEQKPKGEGIPTTAKLKIDEFESNVNEVKDPYPSADFPGDDEEDEPEIPLSPRPRPLAELQLKEKA
VPIPEASSFFIFSPTNKIRVLCHRIVNATWFTNFILLFILLSSAALAAEDPIRADSMRNQILKHFDIGFT
SVFTVEIVLKMTTYGAFLHKGSFCRNYFNMLDLLVVAVSLISMGLESSAISVVKILRVLRVLRPLRAINR
AKGLKHVVQCMFVAISTIGNIVLVTTLLQFMFACIGVQLFKGKFFRCTDLSKMTEEECRGYYYVYKDGDP
MQIELRHREWVHSDFHFDNVLSAMMSLFTVSTFEGWPQLLYKAIDSNAEDVGPIYNNRVEMAIFFIIYII
LIAFFMMNIFVGFVIVTFQEQGETEYKNCELDKNQRQCVQYALKARPLRCYIPKNPYQYQVWYIVTSSYF
EYLMFALIMLNTICLGMQHYNQSEQMNHISDILNVAFTIIFTLEMILKLMAFKARGYFGDPWNVFDFLIV
IGSIIDVILSEIDTFLASSGGLYCLGGGCGNVDPDESARISSAFFRLFRVMRLIKLLSRAEGVRTLLWTF
IKSFQALPYVALLIVMLFFIYAVIGMQMFGKIALVDGTQINRNNNFQTFPQAVLLLFRCATGEAWQEILL
ACSYGKLCDPESDYAPGEEYTCGTNFAYYYFISFYMLCAFLVINLFVAVIMDNFDYLTRDWSILGPHHLD
EFKAIWAEYDPEAKGRIKHLDVVTLLRRIQPPLGFGKFCPHRVACKRLVGMNMPLNSDGTVTFNATLFAL
VRTALKIKTEGNFEQANEELRAIIKKIWKRTSMKLLDQVIPPIGDDEVTVGKFYATFLIQEHFRKFMKRQ
EEYYGYRPKKDIVQIQAGLRTIEEEAAPEICRTVSGDLAAEEELERAMVEAAMEEGIFRRTGGLFGQVDN
FLERTNSLPPVMANQRPLQFAEIEMEEMESPVFLEDFPQDPRINPLARANTNNANANVAYGNSNHSNSHV
FSSVHYEREFPEETETPATRGRALGQPCRVLGPHSKPCVEMLKGLLTQRAMPRGQAPPAPCQCPRVESSM
PEDRKSSTPGSLHEETPHSRSTRENTSRCSAPATALLIQKALVRGGLGTLAADANFIMATGQALADACQM
EPEEVEIMATELLKGREAPEGMASSLGCLNLGSSLGSLDQHQGSQETLIPPRL
Cav1.2 (CACNA1C)
The amino acid sequence of human Cav1.2 (encoded by CACNA1C) is set forth in SEQ ID NO: 2:
(SEQ ID NO: 2)
MVNENTRMYIPEENHQGSNYGSPRPAHANMNANAAAGLAPEHIPTPGAALSWQAAIDAARQAKLMGSAGN
ATISTVSSTQRKRQQYGKPKKQGSTTATRPPRALLCLTLKNPIRRACISIVEWKPFEIIILLTIFANCVA
LAIYIPFPEDDSNATNSNLERVEYLFLIIFTVEAFLKVIAYGLLFHPNAYLRNGWNLLDFIIVVVGLFSA
ILEQATKADGANALGGKGAGFDVKALRAFRVLRPLRLVSGVPSLQVVLNSIIKAMVPLLHIALLVLFVII
IYAIIGLELFMGKMHKTCYNQEGIADVPAEDDPSPCALETGHGRQCQNGTVCKPGWDGPKHGITNFDNFA
FAMLTVFQCITMEGWTDVLYWVNDAVGRDWPWIYFVTLIIIGSFFVLNLVLGVLSGEFSKEREKAKARGD
FQKLREKQQLEEDLKGYLDWITQAEDIDPENEDEGMDEEKPRNMSMPTSETESVNTENVAGGDIEGENCG
ARLAHRISKSKFSRYWRRWNRFCRRKCRAAVKSNVFYWLVIFLVFLNTLTIASEHYNQPNWLTEVQDTAN
KALLALFTAEMLLKMYSLGLQAYFVSLFNRFDCFVVCGGILETILVETKIMSPLGISVLRCVRLLRIFKI
TRYWNSLSNLVASLLNSVRSIASLLLLLFLFIIIFSLLGMQLFGGKFNFDEMQTRRSTFDNFPQSLLTVF
QILTGEDWNSVMYDGIMAYGGPSFPGMLVCIYFIILFICGNYILLNVFLAIAVDNLADAESLTSAQKEEE
EEKERKKLARTASPEKKQELVEKPAVGESKEEKIELKSITADGESPPATKINMDDLQPNENEDKSPYPNP
ETTGEEDEEEPEMPVGPRPRPLSELHLKEKAVPMPEASAFFIFSSNNRFRLQCHRIVNDTIFTNLILFFI
LLSSISLAAEDPVQHTSFRNHILFYEDIVFTTIFTIEIALKILGNADYVFTSIFTLEIILKMTAYGAFLH
KGSFCRNYFNILDLLVVSVSLISFGIQSSAINVVKILRVLRVLRPLRAINRAKGLKHVVQCVFVAIRTIG
NIVIVTTLLQFMFACIGVQLFKGKLYTCSDSSKQTEAECKGNYITYKDGEVDHPIIQPRSWENSKFDFDN
VLAAMMALFTVSTFEGWPELLYRSIDSHTEDKGPIYNYRVEISIFFIIYIIIIAFFMMNIFVGFVIVTFQ
EQGEQEYKNCELDKNQRQCVEYALKARPLRRYIPKNQHQYKVWYVVNSTYFEYLMFVLILLNTICLAMQH
YGQSCLFKIAMNILNMLFTGLFTVEMILKLIAFKPKGYFSDPWNVFDFLIVIGSIIDVILSETNHYFCDA
WNTFDALIVVGSIVDIAITEVNPAEHTQCSPSMNAEENSRISITFFRLFRVMRLVKLLSRGEGIRTLLWT
FIKSFQALPYVALLIVMLFFIYAVIGMQVFGKIALNDTTEINRNNNFQTFPQAVLLLFRCATGEAWQDIM
LACMPGKKCAPESEPSNSTEGETPCGSSFAVFYFISFYMLCAFLIINLFVAVIMDNFDYLTRDWSILGPH
HLDEFKRIWAEYDPEAKGRIKHLDVVTLLRRIQPPLGFGKLCPHRVACKRLVSMNMPLNSDGTVMFNATL
FALVRTALRIKTEGNLEQANEELRAIIKKIWKRTSMKLLDQVVPPAGDDEVTVGKFYATFLIQEYFRKFK
KRKEQGLVGKPSQRNALSLQAGLRTLHDIGPEIRRAISGDLTAEEELDKAMKEAVSAASEDDIFRRAGGL
FGNHVSYYQSDGRSAFPQTFTTQRPLHINKAGSSQGDTESPSHEKLVDSTFTPSSYSSTGSNANINNANN
TALGRLPRPAGYPSTVSTVEGHGPPLSPAIRVQEVAWKLSSNRCHSRESQAAMAGQEETSQDETYEVKMN
HDTEACSEPSLLSTEMLSYQDDENRQLTLPEEDKRDIRQSPKRGFLRSASLGRRASFHLECLKRQKDRGG
DISQKTVLPLHLVHHQALAVAGLSPLLQRSHSPASFPRPFATPPATPGSRGWPPQPVPTLRLEGVESSEK
LNSSFPSIHCGSWAETTPGGGGSSAARRVRPVSLMVPSQAGAPGRQFHGSASSLVEAVLISEGLGQFAQD
PKFIEVTTQELADACDMTIEEMESAADNILSGGAPQSPNGALLPFVNCRDAGQDRAGGEEDAGCVRARGR
PSEEELQDSRVYVSSL
Cav1.3 (CACNA1D)
The amino acid sequence of human Cav1.3 (encoded by CACNA1D) is set forth in SEQ ID NO: 3:
(SEQ ID NO: 3)
MMMMMMMKKMQHQRQQQADHANEANYARGTRLPLSGEGPTSQPNSSKQTVLSWQAAIDAARQAKAAQTMS
TSAPPPVGSLSQRKRQQYAKSKKQGNSSNSRPARALFCLSLNNPIRRACISIVEWKPFDIFILLAIFANC
VALAIYIPFPEDDSNSTNHNLEKVEYAFLIIFTVETFLKIIAYGLLLHPNAYVRNGWNLLDEVIVIVGLF
SVILEQLTKETEGGNHSSGKSGGFDVKALRAFRVLRPLRLVSGVPSLQVVLNSIIKAMVPLLHIALLVLF
VIIIYAIIGLELFIGKMHKTCFFADSDIVAEEDPAPCAFSGNGRQCTANGTECRSGWVGPNGGITNFDNF
AFAMLTVFQCITMEGWTDVLYWVNDAIGWEWPWVYFVSLIILGSFFVLNLVLGVLSGEFSKEREKAKARG
DFQKLREKQQLEEDLKGYLDWITQAEDIDPENEEEGGEEGKRNTSMPTSETESVNTENVSGEGENRGCCG
SLWCWWRRRGAAKAGPSGCRRWGQAISKSKLSRRWRRWNRFNRRRCRAAVKSVTFYWLVIVLVFLNTLTI
SSEHYNQPDWLTQIQDIANKVLLALFTCEMLVKMYSLGLQAYFVSLFNRFDCFVVCGGITETILVELEIM
SPLGISVFRCVRLLRIFKVTRHWTSLSNLVASLLNSMKSIASLLLLLFLFIIIFSLLGMQLFGGKFNFDE
TQTKRSTFDNFPQALLTVFQILTGEDWNAVMYDGIMAYGGPSSSGMIVCIYFIILFICGNYILLNVFLAI
AVDNLADAESLNTAQKEEAEEKERKKIARKESLENKKNNKPEVNQIANSDNKVTIDDYREEDEDKDPYPP
CDVPVGEEEEEEEEDEPEVPAGPRPRRISELNMKEKIAPIPEGSAFFILSKINPIRVGCHKLINHHIFTN
LILVFIMLSSAALAAEDPIRSHSFRNTILGYFDYAFTAIFTVEILLKMTTFGAFLHKGAFCRNYFNLLDM
LVVGVSLVSFGIQSSAISVVKILRVLRVLRPLRAINRAKGLKHVVQCVFVAIRTIGNIMIVTTLLQFMFA
CIGVQLFKGKFYRCTDEAKSNPEECRGLFILYKDGDVDSPVVRERIWQNSDFNFDNVLSAMMALFTVSTF
EGWPALLYKAIDSNGENIGPIYNHRVEISIFFIIYIIIVAFFMMNIFVGFVIVTFQEQGEKEYKNCELDK
NQRQCVEYALKARPLRRYIPKNPYQYKFWYVVNSSPFEYMMFVLIMLNTLCLAMQHYEQSKMFNDAMDIL
NMVFTGVFTVEMVLKVIAFKPKGYFSDAWNTFDSLIVIGSIIDVALSEADPTESENVPVPTATPGNSEES
NRISITFFRLFRVMRLVKLLSRGEGIRTLLWTFIKSFQALPYVALLIAMLFFIYAVIGMQMFGKVAMRDN
NQINRNNNFQTFPQAVLLLFRCATGEAWQEIMLACLPGKLCDPESDYNPGEEYTCGSNFAIVYFISFYML
CAFLIINLFVAVIMDNFDYLTRDWSILGPHHLDEFKRIWSEYDPEAKGRIKHLDVVTLLRRIQPPLGFGK
LCPHRVACKRLVAMNMPLNSDGTVMFNATLFALVRTALKIKTEGNLEQANEELRAVIKKIWKKTSMKLLD
QVVPPAGDDEVTVGKFYATFLIQDYFRKFKKRKEQGLVGKYPAKNTTIALQAGLRTLHDIGPEIRRAISC
DLQDDEPEETKREEEDDVFKRNGALLGNHVNHVNSDRRDSLQQTNTTHRPLHVQRPSIPPASDTEKPLFP
PAGNSVCHNHHNHNSIGKQVPTSTNANLNNANMSKAAHGKRPSIGNLEHVSENGHHSSHKHDREPQRRSS
VKRTRYYETYIRSDSGDEQLPTICREDPEIHGYFRDPHCLGEQEYFSSEECYEDDSSPTWSRQNYGYYSR
YPGRNIDSERPRGYHHPQGFLEDDDSPVCYDSRRSPRRRLLPPTPASHRRSSFNFECLRRQSSQEEVPSS
PIFPHRTALPLHLMQQQIMAVAGLDSSKAQKYSPSHSTRSWATPPATPPYRDWTPCYTPLIQVEQSEALD
QVNGSLPSLHRSSWYTDEPDISYRTFTPASLTVPSSFRNKNSDKQRSADSLVEAVLISEGLGRYARDPKF
VSATKHEIADACDLTIDEMESAASTLLNGNVRPRANGDVGPLSHRQDYELQDFGPGYSDEEPDPGRDEED
LADEMICITTL
Cav1.4 (CACNA1F)
The amino acid sequence of human Cav1.4 (encoded by CACNA1F) is set forth in SEQ ID NO: 4:
(SEQ ID NO: 4)
MSESEGGKDTTPEPSPANGAGPGPEWGLCPGPPAVEGESSGASGLGTPKRRNQHSKHKTVAVASAQRSPR
ALFCLTLANPLRRSCISIVEWKPEDILILLTIFANCVALGVYIPFPEDDSNTANHNLEQVEYVELVIFTV
ETVLKIVAYGLVLHPSAYIRNGWNLLDFIIVVVGLFSVLLEQGPGRPGDAPHTGGKPGGFDVKALRAFRV
LRPLRLVSGVPSLHIVLNSIMKALVPLLHIALLVLFVIIIYAIIGLELFLGRMHKTCYFLGSDMEAEEDP
SPCASSGSGRACTLNQTECRGRWPGPNGGITNFDNFFFAMLTVFQCVTMEGWTDVLYWMQDAMGYELPWV
YFVSLVIFGSFFVLNLVLGVLSGEFSKEREKAKARGDFQKQREKQQMEEDLRGYLDWITQAEELDMEDPS
ADDNLGSMAEEGRAGHRPQLAELTNRRRGRLRWFSHSTRSTHSTSSHASLPASDTGSMTETQGDEDEEEG
ALASCTRCLNKIMKTRVCRRLRRANRVLRARCRRAVKSNACYWAVLLLVFLNTLTIASEHHGQPVWLTQI
QEYANKVLLCLFTVEMLLKLYGLGPSAYVSSFFNRFDCFVVCGGILETTLVEVGAMQPLGISVLRCVRLL
RIFKVTRHWASLSNLVASLLNSMKSIASLLLLLFLFIIIFSLLGMQLFGGKFNFDQTHTKRSTFDTFPQA
LLTVFQILTGEDWNVVMYDGIMAYGGPFFPGMLVCIYFIILFICGNYILLNVFLAIAVDNLASGDAGTAK
DKGGEKSNEKDLPQENEGLVPGVEKEEEEGARREGADMEEEEEEEEEEEEEEEEEGAGGVELLQEVVPKE
KVVPIPEGSAFFCLSQTNPLRKGCHTLIHHHVFTNLILVFIILSSVSLAAEDPIRAHSFRNHILGYFDYA
FTSIFTVEILLKMTVFGAFLHRGSFCRSWFNMLDLLVVSVSLISFGIHSSAISVVKILRVLRVLRPLRAI
NRAKGLKHVVQCVFVAIRTIGNIMIVTTLLQFMFACIGVQLFKGKFYTCTDEAKHTPQECKGSFLVYPDG
DVSRPLVRERLWVNSDFNFDNVLSAMMALFTVSTFEGWPALLYKAIDAYAEDHGPIYNYRVEISVFFIVY
IIIIAFFMMNIFVGFVIITFRAQGEQEYQNCELDKNQRQCVEYALKAQPLRRYIPKNPHQYRVWATVNSA
AFEYLMFLLILLNTVALAMQHYEQTAPFNYAMDILNMVFTGLFTIEMVLKIIAFKPKHYFTDAWNTFDAL
IVVGSIVDIAVTEVNNGGHLGESSEDSSRISITFFRLFRVMRLVKLLSKGEGIRTLLWTFIKSFQALPYV
ALLIAMIFFIYAVIGMQMFGKVALQDGTQINRNNNFQTFPQAVLLLFRCATGEAWQEIMLASLPGNRCDP
ESDFGPGEEFTCGSNFAIAYFISFFMLCAFLIINLFVAVIMDNFDYLTRDWSILGPHHLDEFKRIWSEYD
PGAKGRIKHLDVVALLRRIQPPLGFGKLCPHRVACKRLVAMNMPLNSDGTVTFNATLFALVRTSLKIKTE
GNLEQANQELRIVIKKIWKRMKQKLLDEVIPPPDEEEVTVGKFYATFLIQDYFRKFRRRKEKGLLGNDAA
PSTSSALQAGLRSLQDLGPEMRQALTCDTEEEEEEGQEGVEEEDEKDLETNKATMVSQPSARRGSGISVS
LPVGDRLPDSLSFGPSDDDRGTPTSSQPSVPQAGSNTHRRGSGALIFTIPEEGNSQPKGTKGQNKQDEDE
EVPDRLSYLDEQAGTPPCSVLLPPHRAQRYMDGHLVPRRRLLPPTPAGRKPSFTIQCLQRQGSCEDLPIP
GTYHRGRNSGPNRAQGSWATPPQRGRLLYAPLLLVEEGAAGEGYLGRSSGPLRTFTCLHVPGTHSDPSHG
KRGSADSLVEAVLISEGLGLFARDPRFVALAKQEIADACRLTLDEMDNAASDLLAQGTSSLYSDEESILS
RFDEEDLGDEMACVHAL

### Cav1.4 (CACNA1F) Mutant I745T

The amino acid sequence of the human CACNA1F I745T mutant is set forth in SEQ ID NO: 5. This mutation is also referred to in the literature as I756T (e.g., Williams, 2020). The mutated residue is highlighted in bold and underlined.

(SEQ ID NO: 5)
MSESEGGKDTTPEPSPANGAGPGPEWGLCPGPPAVEGESSGASGLGTPKR
RNQHSKHKTVAVASAQRSPRALFCLTLANPLRRSCISIVEWKPFDILILL
TIFANCVALGVYIPFPEDDSNTANHNLEQVEYVFLVIFTVETVLKIVAYG
LVLHPSAYIRNGWNLLDFIIVVVGLFSVLLEQGPGRPGDAPHTGGKPGGF
DVKALRAFRVLRPLRLVSGVPSLHIVLNSIMKALVPLLHIALLVLFVIII
YAIIGLELFLGRMHKTCYFLGSDMEAEEDPSPCASSGSGRACTLNQTECR
GRWPGPNGGITNFDNFFFAMLTVFQCVTMEGWTDVLYWMQDAMGYELPWV
YFVSLVIFGSFFVLNLVLGVLSGEFSKEREKAKARGDFQKQREKQQMEED
LRGYLDWITQAEELDMEDPSADDNLGSMAEEGRAGHRPQLAELTNRRRGR
LRWFSHSTRSTHSTSSHASLPASDTGSMTETQGDEDEEEGALASCTRCLN
KIMKTRVCRRLRRANRVLRARCRRAVKSNACYWAVLLLVFLNTLTIASEH
HGQPVWLTQIQEYANKVLLCLFTVEMLLKLYGLGPSAYVSSFFNRFDCFV
VCGGILETTLVEVGAMQPLGISVLRCVRLLRIFKVTRHWASLSNLVASLL
NSMKSIASLLLLLFLFIIIFSLLGMQLFGGKFNFDQTHTKRSTFDTFPQA
LLTVFQILTGEDWNVVMYDGIMAYGGPFFPGMLVCIYFIILFICGNYILL
NVFLATAVDNLASGDAGTAKDKGGEKSNEKDLPQENEGLVPGVEKEEEEG
ARREGADMEEEEEEEEEEEEEEEEEGAGGVELLQEVVPKEKVVPIPEGSA
FFCLSQTNPLRKGCHTLIHHHVFTNLILVFIILSSVSLAAEDPIRAHSFR
NHILGYFDYAFTSIFTVEILLKMTVFGAFLHRGSFCRSWFNMLDLLVVSV
SLISFGIHSSAISVVKILRVLRVLRPLRAINRAKGLKHVVQCVFVAIRTI
GNIMIVTTLLQFMFACIGVQLFKGKFYTCTDEAKHTPQECKGSFLVYPDG
DVSRPLVRERLWVNSDFNFDNVLSAMMALFTVSTFEGWPALLYKAIDAYA
EDHGPIYNYRVEISVFFIVYIIIIAFFMMNIFVGFVIITFRAQGEQEYQN
CELDKNQRQCVEYALKAQPLRRYIPKNPHQYRVWATVNSAAFEYLMFLLI
LLNTVALAMQHYEQTAPFNYAMDILNMVFTGLFTIEMVLKIIAFKPKHYF
TDAWNTFDALIVVGSIVDIAVTEVNNGGHLGESSEDSSRISITFFRLFRV
MRLVKLLSKGEGIRTLLWTFIKSFQALPYVALLIAMIFFIYAVIGMQMFG
KVALQDGTQINRNNNFQTFPQAVLLLFRCATGEAWQEIMLASLPGNRCDP
ESDFGPGEEFTCGSNFAIAYFISFFMLCAFLIINLFVAVIMDNEDYLTRD
WSILGPHHLDEFKRIWSEYDPGAKGRIKHLDVVALLRRIQPPLGFGKLCP
HRVACKRLVAMNMPLNSDGTVTFNATLFALVRTSLKIKTEGNLEQANQEL
RIVIKKIWKRMKQKLLDEVIPPPDEEEVTVGKFYATFLIQDYFRKFRRRK
EKGLLGNDAAPSTSSALQAGLRSLQDLGPEMRQALTCDTEEEEEEGQEGV
EEEDEKDLETNKATMVSQPSARRGSGISVSLPVGDRLPDSLSFGPSDDDR
GTPTSSQPSVPQAGSNTHRRGSGALIFTIPEEGNSQPKGTKGQNKQDEDE
EVPDRLSYLDEQAGTPPCSVLLPPHRAQRYMDGHLVPRRRLLPPTPAGRK
PSFTIQCLQRQGSCEDLPIPGTYHRGRNSGPNRAQGSWATPPQRGRLLYA
PLLLVEEGAAGEGYLGRSSGPLRTFTCLHVPGTHSDPSHGKRGSADSLVE
AVLISEGLGLFARDPRFVALAKQEIADACRLTLDEMDNAASDLLAQGTSS
LYSDEESILSRFDEEDLGDEMACVHAL

### SUMMARY OF THE FIGURES

Embodiments and experiments illustrating the principles of the invention will now be discussed with reference to the accompanying figures.

FIG. 1. L-type Cav channel agonists increase Gli1 expression and enhance specific cell killing by murine CD8+ CTLs. CD8⁺ T cells were isolated from the spleen and inguinal lymph nodes of Rag2^{-/-} OT-I mice, and CTLs were generated. (A) Cells were restimulated on day 9/10 for 3 hours using plate-bound anti-CD3ε in the presence of indicated concentrations of FPL 64176 or carrier control. RNA was extracted and Gli1 expression was assessed by qRT-PCR. Data is n=3 independent experiments. us=unstimulated cells. (B) An LDH cytotoxicity assay was performed using OT-I CTLs. On day 7, CTLs were co-cultured with Ova peptide-pulsed EL-4 cells in the presence of 10 μM FPL 64176 (squares) or carrier control (circles). Data is n=4 independent experiments. (C) Updated experiment of (A). Data is now n=4 independent experiments. Data is normalised to Tbp as a reference gene. 0h=unstimulated cells. (D) Same data as (B) represented differently. Left panel shows a representative killing assay. Right panel shows quantification of n=4 independent experiments normalised to killing of carrier-treated cells at a 10:1 effector:target ratio. Error bars indicate SD. p values were calculated using an unpaired two-tailed Student's t test (C) or a two-way ANOVA (B/D). * indicates p<0.05, ** indicates p<0.01, **** indicates p<0.0001.

FIG. 2. FPL 64176 but not (R)-Baclofen nor Nefiracetam enhance Gli1 expression in murine CD8+ CTLs. Naïve CD8⁺ T cells were isolated from the spleens of OT-I Rag2^{-/-} mice and CTLs were generated. Cells were restimulated using an anti-CD3ε mAb in the presence indicated concentrations of (A) FPL 64176, (B) (R)-Baclofen or (C) Nefiracetam (or carrier control). CTLs were pre-incubated with the relevant pharmacological agents for 40-50 mins prior to stimulation (ensuring sufficient time for the agents, e.g., FPL 64176, to enter the cell. Gli1 expression was assessed by qRT-PCR. Data is n=2 independent experiments. Error bars indicate SD.

FIG. 3. L-type Cav channel agonists increase expression of GLI1 and enhance specific cell killing by human CD8+ CTLs. Naïve human CD8+ T cells were purified from the buffy coat layer of blood isolated from healthy donors, and were stimulated using ImmunoCult™ CD3/CD28/CD2 human T-Cell Activator and again restimulated at day 10 for 3 days. (A) Human CTLs were restimulated on day 15-20 for 24 h with plate-bound anti-CD3 in the presence of 10 μM of FPL64176 or carrier control before RNA was extracted for qRT-PCR analysis. n=1 healthy donor. TBP is used as a reference gene. (B) Updated graph of (A) showing n=3 independent experiments/healthy donors. Gli1 expression is normalised to carrier. (C) Between day 16 and 24, CD8+ T cells were co-cultured with P815 target cells at indicated effector to target ratios and subjected to a VITAL cytotoxicity assay in the presence of 10 μM FPL64176 (squares) or carrier control (circles). (D) Same data as (C) represented differently. Left panel shows a representative killing assay. Right panel shows quantification of n=4 experiments with 4 different healthy donors normalised to killing of carrier-treated cells at a 10:1 effector:target ratio. Error bars indicate SD. p values were calculated using an unpaired two-tailed Student's t test (B) or a two-way ANOVA (C/D). * indicates p<0.05, ** indicates p<0.01, **** indicates p<0.0001.

FIG. 4. Gli1 is important for specific tumour cell killing by murine NK cells. (A) NK cells were isolated from WT C57BL/6 mice and were treated using either the Gli1/Gli2 inhibitor GANT61 (squares) or carrier control (circles). LDH killing assays (left panel) and VITAL assays (right panel) were performed to assess specific cell killing of NK-sensitive RMA/S tumour cells. Left panel: Representative data of n=3 independent experiments. Right panel: data from 2 individual mice per genotype. (B) To specifically investigate the contribution of Gli1 towards NK cell effector function, LDH killing assays (left panel) and VITAL assays (right panel) were performed using NK cells isolated from both WT (circles) and Gli1 knockout (KO, squares) mice. Left panel: representative data from n=2 independent experiments. Right panel: data from 2 individual mice per genotype.

FIG. 5. L-type Cav channel agonists increase GLI1 expression in the human NK cell line NK-92 and promote specific tumour cell killing. (A) NK-92 cells were stimulated using an anti-NKp46 mAb for 4 hours in the presence of 10 μM FPL 64176, and GLI1 expression was assessed using qRT-PCR. Data is n=4 independent experiments. (B) NK-92 cells were stimulated using an anti-NKp46 mAb for 4 hours in the presence of 100 μM Bay K8644, and GLI1 expression was assessed using qRT-PCR. Data is n=3 independent experiments. (C) An LDH assay was performed to assess specific cell killing of K562 target cells at indicated effector to target ratios in the presence of 10 μM FPL 64176 (right-hand columns) or carrier control (left-hand columns). Error bars indicate SD. Data is from n=3 independent experiments.

FIG. 6. Mutations leading to a gain-of-function Hh phenotype with increased GLI1 expression and enhanced specific tumour cell killing by human NK cells. NK cells were purified from the buffy coat layer of blood isolated from patients with Gorlin syndrome and healthy control individuals and were cultured for 2 days in complete RPMI+20 ng/ml hIL-15. (A) On day 2, NK cells were analysed for expression of GLI1 by qRT-PCR. (B) A VITAL assay was performed to assess specific killing of K562 target cells. Data is shown in replicate form, representing NK cells derived from three individual Gorlin syndrome patients (squares) and from three healthy control individuals (circles).

FIG. 7. NK-92 cells overexpressing the I756T Cav1.4 gain-of-function mutation show enhanced tumour cell killing. NK-92 cells (Parental) were engineered to stably express empty vector (EV), Cav1.4 (WT), Cav1.4 harbouring the gain-of-function mutation I756T and the loss-of-function mutation S229P, respectively. Killing of K562 tumour cells was assessed after 24 h by LDH killing assay. Stable expression of WT, I756T (GOF), or S229P (LOF) Cav1.4 in NK-92 cells does not affect cell survival and proliferation. I756T (GOF) Cav1.4 can also be successfully expressed in human CD8 T cells.

FIG. 8. Gli1 mediates specific cell killing by murine cytotoxic CD4+ T cells. Cytotoxic CD4+ T cells were generated from OT-II mice and treated with 5 μM Gli1/Gli2 inhibitor GANT61 (grey) or carrier control (black) for 24 hours before an LDH assay was performed to assess specific cell killing. Data is from n=3 independent experiments.

FIG. 9. GzmB content and IFNγ secretion are unaffected by Gli inhibition in cytotoxic CD4+ T cells. Cytotoxic CD4+ cells were generated from OT-II mice and treated with 5 μM GANT61 or carrier control on day 7 for 24 h prior to flow cytometric analysis of Granzyme B (GzmB) and IFNγ levels on day 8. (A) Representative flow cytometry plots in unstimulated (top panel) or restimulated CD4+ T cells (bottom panel). (B) Quantification of steady state GzmB levels and IFNγ levels upon restimulation. Data is from n=3 independent experiments.

FIG. 10. GzmB content and IFNγ secretion are unaffected by Gli inhibition in NK cells. NK cells were isolated from C57BL/6 mice and treated with 10 μM GANT61 or carrier control (EtOH) overnight (˜18 hours) prior to analysis of intracellular granzyme B (GzmB) and IFNγ expression by flow cytometry. (A) Representative flow cytometry plots. (B) Quantification of flow cytometry plots. Data is from n=2 mice, 1 independent experiment.

FIG. 11. FPL 64176 treatment does not impair viability or proliferation of naïve and cytotoxic CD8+ T cells, does not affect memory phenotypes, and enhances CTL killing via extracellular Ca²⁺. Naïve CD8+ T cells were stimulated with plate-bound anti-CD3/CD28 antibodies for 24 h in the presence of 10 μM FPL64176 or carrier control before flow cytometric analysis (FIG. 11A-D). (A) Quantitative analysis of live cell numbers. (B) Percentage of cells positive for the apoptotic marker Apotracker™ Green. (C) Percentage of cells positive for cell viability dye DAPI. (D) Analysis of T cell subsets based on the expression of CD44 and CD62L: CD44− CD62L+ (naïve), CD44+ CD62L+ (central memory), and CD44+ CD62L− (effector memory). (FIG. 11E-G) CTLs were restimulated on day 10 with plate-bound anti-CD3 antibody for 24 h in the presence of 10 μM FPL64176 or carrier control before flow cytometric analysis. (E) Quantitative analysis of live cell numbers. (F) Percentage of cells positive for the apoptotic marker Apotracker™ Green. (G) Percentage of cells positive for the cell viability dye DAPI. (H) On day 7-8 post stimulation, murine CTLs were co-cultured with ovalbumin-pulsed EL-4 target cells at the indicated effector to target ratios in the presence of 10 μM FPL64176 and/or 1.25 mM BAPTA and their respective carrier controls and subjected to an LDH cytotoxicity assay for 3 h. n=4 independent experiments normalised to killing of carrier-treated cells at a 10:1 effector:target cell ratio. Error bars indicate SD.

FIG. 12. GLI1 is important for tumour cell killing of human Vγ9Vδ2 gammadelta T cells and treatment with L-type voltage gated Ca2+ channel agonist FPL 64176 increases killing capacity. PBMCs were isolated from Buffy Coats of healthy donors using SepMate PBMC Isolation Tubes. Vγ9Vδ2 expansion was induced by plating PBMCs at 1×10⁶ concentration with 1 μM of Zoledronate in the presence of 100 IU/mL IL-2 and 120 IU/mL IL-15. Cells were split every 2-3 days from day 6 until day 20. Killing assays were conducted between day 16-20. (A) Vγ9Vδ2 T lymphocytes were incubated with Gli1/2 inhibitor GANT61 or EToH (carrier control) overnight and subsequently co-cultured with K562 target cells for 24 h at the indicated effector to target ratios and subjected to a flow-cytometry based killing assay. n=1 individual donor. (B) Vγ9Vδ2 cells were pre-incubated with 10 μM FPL 64176 or EToH (carrier control) before being co-cultured with K562 target cells at indicated effector to target ratios and subjected to a flowcytometric cytotoxicity assay in the presence of 10 μM FPL 64176 or EToH (carrier control). Killing was assessed after 24 hours. Tumour cell killing normalised to carrier treated cells at a 10:1 effector:target ratio. n=4 individual healthy donors from 2 independent experiments.

FIG. 13. Treatment of tumour-specific CD8 T cells with Cav channel agonist FPL 64176 before adoptive transfer into tumour-bearing mice leads to increased tumour cell death in vivo. Cytotoxic OTI T cells were re-stimulated in vitro in the presence of FPL 64176 or carrier control before adoptive transfer into MC38-Ova tumour bearing Rag2 KO mice via tail vein injection. 24 hours after the adoptive transfer, tumours were excised and analysed by immunohistochemistry for Cleaved Caspase 3 (A). 3 mice per condition, % CC3 over whole tumour area from all mice. (B) Repeat experiment with 3-4 mice per condition. Each data point represents the average CC3 staining of 3 separate tumour sections. Overall CC3 staining (top panel) and weak, moderate, and strong CC3 staining (bottom row), respectively, are shown. FPL treatment may not affect effector and central memory phenotypes in tumour, spleen and draining lymph node in vivo.

FIG. 14. L-type voltage gated Ca²⁺ channel agonist FPL 64176 accelerates initiation of Ca²⁺ flux after TCR crosslinking and specifically increases Cav-mediated Ca²⁺ flux in murine CD8⁺ cytotoxic T lymphocytes. Murine CD8⁺ CTLs were loaded with Calcium Sensor Dye eFluor™ 514, anti-CD3ε antibodies and calcium channel inhibitors/agonist. Other Calcium channels expressed in CD8 T cells were inhibited: “Block”=BTP2 inhibits CRAC channels downstream of the TCR, and BCTC inhibits TRPV channels. All inhibitors at the concentration used are non-toxic to T cells (data not shown). T cell receptor (TCR) crosslinking was induced 30 s after the start of sample acquisition by adding antibodies to crosslink the TCR. Arrows indicate the initiation of the calcium flux following TCR crosslinking. The line indicates the baseline fluorescence of the Calcium Sensor Dye eFluor™ 514. Data shown is representative of two independent experiments.

FIG. 15. GLI inhibitor GANT61 reduces tumour cell killing of human CD8+ cytotoxic T lymphocytes. PBMCs were isolated from Buffy Coats of healthy donors using SepMate PBMC Isolation Tubes. CD8+ T cells were isolated using negative selection (Miltenyi Biotec, CD8+ T Cell Isolation Kit) and plated with ImmunoCult™ Human CD3/CD28/CD2 T Cell Activator at 25 μl/mL in TexMACS cell culture media supplemented with 1001 U/mL human IL-2 and 100 U/ml Penicillin/Streptomycin. Human CTLs were incubated with Gli1/2 inhibitor GANT61 or EToH (carrier control) overnight and subsequently co-cultured with anti-CD3ε pulsed P815 target cells for 3 h at the indicated effector to target ratios and subjected to an LDH cytotoxicity assay. (A) Representative graph, (B) Quantification of N=3 donors normalised to killing of carrier treated cells at a 10:1 effector:target ratio. Error bars indicate SD. n=3 donors from 2 independent experiments.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Cav Channels

The voltage-gated calcium channels (Caᵥs) form a family of 10 members, classified according to expression of the pore-forming α1-subunit. These 10 members are grouped across 3 subfamilies: Caᵥ1, Caᵥ2 and Caᵥ3. The Cav1 subfamily is also known as the L-type Cav family, and includes Caᵥ1.1 (α_{1S}), Caᵥ1.2 (α_{1C}), Caᵥ1.3 (α_{1D}) and Caᵥ1.4 (α_{1F}). The ‘L’ denotes the “long-lasting currents” associated with this subfamily. The present invention relates to L-type Caᵥs.

### Immune Cells

The present invention provides an engineered immune cell platform. It is envisaged that the engineered immune cell may be any cytotoxic lymphocyte. For example, the engineered immune cell may be a CD8+ T cell, a CD4+ T cell, or an NK cell. The T cell may be a gamma delta T cell. The inventors hypothesise that all cytotoxic lymphocytes rely on Gli1 for their cell killing function. Cytotoxic lymphocytes exert their effects on their target cells by releasing granzymes, which are serine proteases that induce apoptosis in the target cell. Granzymes are packaged inside cytotoxic granules along with perforin, a pore-forming protein that allows the granzymes to enter the target cell. Humans have five granzymes: Granzyme A, Granzyme B, Granzyme H, Granzyme K and Granzyme M.

### Engineering

The engineered immune cell platform may use any suitable genetic engineering technique. The genetic engineering technique may be nuclease-based.

For example, the engineered immune cell may be engineered using CRISPR/Cas9 gene editing. This technique is based on the bacterial antiviral defence system CRISPR-Cas9. The Cas9 nuclease is complexed with a synthetic guide RNA (gRNA) which directs the nuclease to the desired DNA site to be cleaved by the nuclease.

Alternatively, the engineered immune cell may be engineered using prime editing (Anzalone, 2019). This genome editing method uses a catalytically impaired Cas9 endonuclease fused to an engineered reverse transcriptase which has a prime editing guide RNA (pegRNA) that simultaneously specifies the target site and encodes the desired edit. In this way, new genetic information is directly written into the target DNA site.

### Gain-of-Function

“Gain-of-function” (or “gain of function” or “GOF”) as used herein refers to a modification, such as a mutation, that enhances one or more recognised functions of a gene product. In particular, a gain-of-function mutation (GOF) in an L-type Cav channel may be a mutation that causes the L-type Cav channel to exhibit greater Ca²⁺ flux, faster opening, prolonged opening, accelerated initiation of Ca²⁺ flux, slower inactivation kinetics or other change resulting in greater calcium channel activity in vitro or in vivo as compared with a non-mutated L-type Cav channel tested under like conditions. Specific examples of L-type Cav channel GOF mutation are described above (see, e.g., Table 1).

### PTCH1

The protein “protein patched homolog 1” having the human amino acid sequence disclosed at UniProt Q13635 (as at UniProt Release 2023_01 of 22 Feb. 2023) is encoded by the gene PTCH1 (NCBI GeneID: 5727). A number of variants of PTCH1 have been reported as being present in human patients having Gorlin Syndrome. For example, rs1843916565. All of the PTCH1 mutations associated with Gorlin Syndrome as disclosed at UniProt Q13635 (in the section entitled “Disease & Variants” are expressly incorporated herein by reference in their entirety. PTCH1 mutations that give rise to Gorlin Syndrome are considered PTCH1 loss-of-function mutations herein.

### Chimeric Antigen Receptors

Chimeric Antigen Receptors (CARs) are recombinant receptor molecules which provide both antigen-binding and T cell activating functions. CAR structure and engineering is reviewed, for example, in Dotti et al., Immunol Rev (2014) 257(1), which is hereby incorporated by reference in its entirety.

CARs comprise an antigen-binding domain linked to a transmembrane domain and a signalling domain. An optional hinge domain may provide separation between the antigen-binding domain and transmembrane domain, and may act as a flexible linker.

The antigen-binding domain of a CAR may be based on the antigen-binding region of an antibody which is specific for the antigen to which the CAR is targeted. For example, the antigen-binding domain of a CAR may comprise amino acid sequences for the complementarity-determining regions (CDRs) of an antibody which binds specifically to the target protein. The antigen-binding domain of a CAR may comprise or consist of the light chain and heavy chain variable region amino acid sequences of an antibody which binds specifically to the target protein. The antigen-binding domain may be provided as a single chain variable fragment (scFv) comprising the sequences of the light chain and heavy chain variable region amino acid sequences of an antibody. Antigen-binding domains of CARs may target antigen based on other protein:protein interaction, such as ligand:receptor binding; for example an IL-13Rα2-targeted CAR has been developed using an antigen-binding domain based on IL-13 (see e.g. Kahlon et al. 2004 Cancer Res 64 (24): 9160-9166).

The transmembrane domain is provided between the antigen-binding domain and the signalling domain of the CAR. The transmembrane domain provides for anchoring the CAR to the cell membrane of a cell expressing a CAR, with the antigen-binding domain in the extracellular space, and signalling domain inside the cell. Transmembrane domains of CARs may be derived from transmembrane region sequences for CD3-ζ, CD4, CD8 or CD28.

The signalling domain allows for activation of the T cell. The CAR signalling domains may comprise the amino acid sequence of the intracellular domain of CD3-ζ, which provides immunoreceptor tyrosine-based activation motifs (ITAMs) for phosphorylation and activation of the CAR-expressing T cell. Signalling domains comprising sequences of other ITAM-containing proteins have also been employed in CARs, such as domains comprising the ITAM containing region of FcγRI (Haynes et al., 2001 J Immunol 166(1):182-187). CARS comprising a signalling domain derived from the intracellular domain of CD3-ζ are often referred to as first generation CARs.

Signalling domains of CARs may also comprise co-stimulatory sequences derived from the signalling domains of co-stimulatory molecules, to facilitate activation of CAR-expressing T cells upon binding to the target protein. Suitable co-stimulatory molecules include CD28, OX40, 4-1BB, ICOS and CD27. CARs having a signalling domain including additional co-stimulatory sequences are often referred to as second generation CARs.

In some cases CARs are engineered to provide for co-stimulation of different intracellular signalling pathways. For example, signalling associated with CD28 costimulation preferentially activates the phosphatidylinositol 3-kinase (P13K) pathway, whereas the 4-1 BB-mediated signalling is through TNF receptor associated factor (TRAF) adaptor proteins. Signalling domains of CARs therefore sometimes contain co-stimulatory sequences derived from signalling domains of more than one co-stimulatory molecule. CARs comprising a signalling domain with multiple co-stimulatory sequences are often referred to as third generation CARs.

An optional hinge region may provide separation between the antigen-binding domain and the transmembrane domain, and may act as a flexible linker. Hinge regions may be flexible domains allowing the binding moiety to orient in different directions. Hinge regions may be derived from IgG1 or the CH₂CH₃ region of immunoglobulin.

### Adoptive Transfer

In embodiments of the present invention, a method of treatment or prophylaxis may comprise adoptive transfer of immune cells, particularly T cells. Adoptive T cell transfer generally refers to a process by which T cells are obtained from a subject, typically by drawing a blood sample from which T cells are isolated. The T cells are then typically treated or altered in some way, optionally expanded, and then administered either to the same subject or to a different subject. The treatment is typically aimed at providing a T cell population with certain desired characteristics to a subject, or increasing the frequency of T cells with such characteristics in that subject. Adoptive transfer of CAR-T cells is described, for example, in Kalos and June 2013, Immunity 39(1): 49-60, which is hereby incorporated by reference in its entirety.

Embodiments of the present invention may also comprise adoptive transfer of NK cells.

In some embodiments, the subject from which the T cell is isolated is the subject administered with the modified T cell (i.e., adoptive transfer is of autologous T cells). In some embodiments, the subject from which the T cell is isolated is a different subject to the subject to which the modified T cell is administered (i.e., adoptive transfer is of allogeneic T cells).

### T Cell Therapy

T cell therapy can include adoptive T cell therapy, tumour-infiltrating lymphocyte (TIL) immunotherapy, autologous cell therapy, engineered autologous cell therapy (eACT™), and allogeneic T cell transplantation.

The T cells of the immunotherapy can come from any source known in the art. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumours. In addition, the T cells can be derived from one or more T cell lines available in the art. T cells can also be obtained from a unit of blood collected from a subject using any number of techniques known to the skilled artisan, such as FICOLL™ Separation and/or apheresis. Additional methods of isolating T cells for a T cell therapy are disclosed in US2013/0287748, which is herein incorporated by references in its entirety.

### Gli1 Reporter Assays

Any suitable Gli1 reporter assay may be used as described herein. A Gli1 reporter assay may be used to determine stimulation of L-type Cav channel activity. For example, the Gli1 reporter assay may be any assay that detects binding to the Gli1 consensus sequence. Such an assay may employ the use of fluorescent probes or may be a colorimetric assay. The Gli1 reporter assay may detect Gli1 mRNA via qRT-PCR. The GLi1 reporter assay may detect Gli1 protein levels and nuclear translocation via immunofluorescence.

### Cancers

A “cancer” can comprise any one or more of the following: acute lymphocytic leukaemia (ALL), acute myeloid leukaemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukaemia (CLL), chronic myeloid leukaemia (CML), colon and rectal cancer, colon cancer, endometrial cancer, endometrial sarcoma, oesophageal cancer, eye cancer, gallbladder cancer, gastric cancer, gastrointestinal tract cancer, hairy cell leukaemia, head and neck cancer, hepatocellular cancer, Hodgkin's disease, hypopharyngeal cancer, Kaposi's sarcoma, kidney cancer, laryngeal cancer, leukaemia, leukaemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumour, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia and Wilms' tumour.

Cancers may be of a particular type. Examples of types of cancer include astrocytoma, carcinoma (e.g. adenocarcinoma, hepatocellular carcinoma, medullary carcinoma, papillary carcinoma, squamous cell carcinoma), glioma, lymphoma, medulloblastoma, melanoma, myeloma, meningioma, neuroblastoma, sarcoma (e.g. angiosarcoma, chondrosarcoma, osteosarcoma).

In one preferred embodiment, the cancer is a melanoma, lung cancer, colorectal cancers or breast cancer. Preferably, the cancer may also be a lymphoma, leukaemia (including acute lymphoblastic leukaemia and chronic lymphocytic leukaemia) or multiple myeloma.

Therapy for cancers, such as the use of compositions and/or cells of the present invention, may advantageously be combined with other anti-cancer therapies, such as with a checkpoint inhibitor (e.g., anti-PD-1, anti-PD-L1, anti-CTLA4 antibodies, such as Nivolumab, Atezolizumab or Pembrolizumab).

### Autoimmune Diseases

Autoimmune diseases are inflammatory diseases which arise as a result of aberrant activation of the immune response. Without being bound by theory, it is believed that autoimmune diseases may arise from failures in the processes of immune regulation (for example, including failures in Thymic selection) or from persistent activation of the immune response (for example, via the sustained release of damage-associated molecular patterns (DAMPs) or pathogen-associated molecular patterns (PAMPs)).

Autoimmune diseases are often associated with the production of self-reactive autoantibodies, and/or self-reactive lymphocytes, such as autoreactive T cells and B cells.

It is widely known that autoimmune diseases may affect any aspect of the body. The term “autoimmune disease” may be any one or more of the following diseases: acquired aplastic anaemia, acquired haemophilia, primary agammaglobulinemia, alopecia areata, ankylosing spondylitis (AS), anti-NMDA receptor encephalitis, antiphospholipid syndrome (APS) (including catastrophic antiphospholipid syndrome (CAPS)), autoimmune Addison's disease (AAD), autoimmune autonomic ganglionopathy (AAG), autoimmune gastrointestinal dysmotility (AGID), autoimmune encephalitis (including acute disseminated encephalomyelitis (ADEM)) autoimmune gastritis, autoimmune haemolytic anaemia (AIHA), autoimmune hepatitis (AIH), autoimmune myocarditis, autoimmune oophoritis, autoimmune pancreatitis (AIP), autoimmune polyglandular syndromes (including types I, II, & III), autoimmune progesterone dermatitis, Balo disease, Behçet's disease, birdshot uveitis, bullous pemphigoid, celiac disease, chronic inflammatory demyelinating polyneuropathy (CIDP), Churg-Strauss syndrome, Cogan's syndrome, cold agglutinin disease, CREST syndrome, Crohn's disease (CD), cutaneous lupus erythematosus, dermatitis herpetiformis, dermatomyositis, Devic's disease Type 1 diabetes, discoid lupus, eosinophilic fasciitis, Evans syndrome, glomerulonephritis, Goodpasture's syndrome, granulomatosis with polyangiitis (GPA), Graves disease, Guillain-Barré syndrome (GBS), Hashimoto's thyroiditis, Henoch-Schönlein purpura, Hurst's disease, Berger's disease, immune-mediated necrotizing myopathy (IMNM), immune thrombocytopenia (ITP), inflammatory bowel disease (IBD), juvenile idiopathic arthritis, juvenile myositis, Lambert-Eaton myasthenic syndrome (LEMS), linear IgA disease (LAD), lupus nephritis, Ménière's disease, mixed connective tissue disease (MCTD), multiple sclerosis (MS), myasthenia gravis (MG), ocular cicatricial pemphigoid, palindromic rheumatism, paraneoplastic cerebellar degeneration, paraneoplastic pemphigus, paroxysmal nocturnal haemoglobinuria (PNH), Parsonage-Turner syndrome, herpes gestationis, pemphigus foliaceus, pemphigus vulgaris, POEMS syndrome, polyarteritis nodosa, polymyalgia rheumatica, polymyositis, primary biliary cirrhosis (PBC), psoriasis, psoriatic arthritis, pure red cell aplasia (PRCA), Raynaud's syndrome, Reiter's syndrome, relapsing polychondritis, rheumatic fever, rheumatoid arthritis (RA), sarcoidosis, Schmidt syndrome, scleroderma, Sjögren's syndrome, small fibre sensory neuropathy, systemic lupus erythematosus (SLE), ulcerative colitis (UC), undifferentiated connective tissue disease (UCTD) and Vitiligo.

Preferably, the autoimmune disease is rheumatoid arthritis (RA), rheumatoid arthritis (RA), systemic lupus erythematosus (SLE), multiple sclerosis (MS) or Type 1 diabetes.

### Infectious Diseases

Infectious diseases are characterised by infection of a patient with an infectious agent, and are thereby typically associated with activation of the immune response. Infectious agents may include microorganisms such as viruses, bacteria, protozoa, or fungi. Small animals may be considered infectious agents, usually termed ‘parasites. In addition, the term “infectious agent” may also refer to a prion.

Viruses may include hepatitis viruses, adenoviruses, rabies viruses, papillomaviruses, rotaviruses, herpesviruses, cytomegaloviruses, and bacteriophage. In some aspects of the disclosure, the term ‘virus’ may refer to human retroviruses, such as HIV. The term ‘virus’ may also refer to pandemic coronaviruses, including the SARS-CoV-II virus.

Pathogenic bacteria include Staphylococcus, Streptococcus, Neisseria, Clostridium, Bacillus, Listeria, Escherichia, Salmonella, Vibrio, Campylobacter, Bordetella, Haemophilus, Legionella, Helicobacter, Mycobacterium, Treponema, Borrelia, Chlamydia and Rickettsia species. Pathogenic protozoa include Entamoeba, Trichomonas, Leishmania, Chilomonas, Giardia, Isopora, Sarocystis, Nosema, Balantidium, Histomonas, Trypanosoma, Plasmodium, Babesia, Haemoproteus and Eimeria species. Pathogenic fungi include Candida, Pneumocystitis, Histoplasma, Blastomyces, Malassezia and Cryptococcus species. Parasites may include intracellular or extracellular parasites, such as Ancylostoma, Ascaris, Enteroblus, Paragonimus, Schitosoma, Taenia, Trichuris, Wuchereria, Balantidium, Entamoeba, Giardia, Leishmania, Plasmodium, Trichomonas and Trypanosoma species. Infectious diseases caused by prions may include Creutzfeldt-Jakob disease (CJD), variant Creutzfeldt-Jakob disease (CJD), variably protease-sensitive prionopathy (VPSPr), Gerstmann-Sträussler-Scheinker disease (GSS), Kuru and fatal insomnia (FI).

Preferably, the infectious disease is infection with: HIV, SARS-CoV-II, hepatitis C, hepatitis B, human cytomegalovirus or an opportunistic fungal infection such as aspergillosis.

### Subject

The subject to be treated according to the invention may be any animal or human. The subject is preferably mammalian, such as a cat, dog, horse, donkey, sheep, pig, goat, cow, mouse, rat, rabbit or guinea pig, more preferably a human. The subject may be male or female. The subject may be a patient. A subject may have been diagnosed with a disease or condition (such as a cancer requiring treatment, may be suspected of having such a disease or condition, or may be at risk from developing such a disease or condition. The subject may be a juvenile, such as a human child (e.g. a child having a paediatric cancer).

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### Experiments

The following data are provided to demonstrate the efficacy and therapeutic utility of the invention. These data are intended to be illustrative and non-limiting upon the scope of protection.

### BACKGROUND

Hedgehog (Hh) signalling is known to control T cell killing, by regulating formation of the immunological synapse. For example, de la Roche et al. (2013) report that stimulation of the T cell receptor (TCR) in murine CD8+ T cells drives Hh activation, thereby initiating the expression of downstream Hh components, including Gli1. The authors specifically identify a role for Hh signalling in centrosome polarisation and actin reorganisation in CTLs, controlling formation of the immunological synapse and therefore CTL effector function.

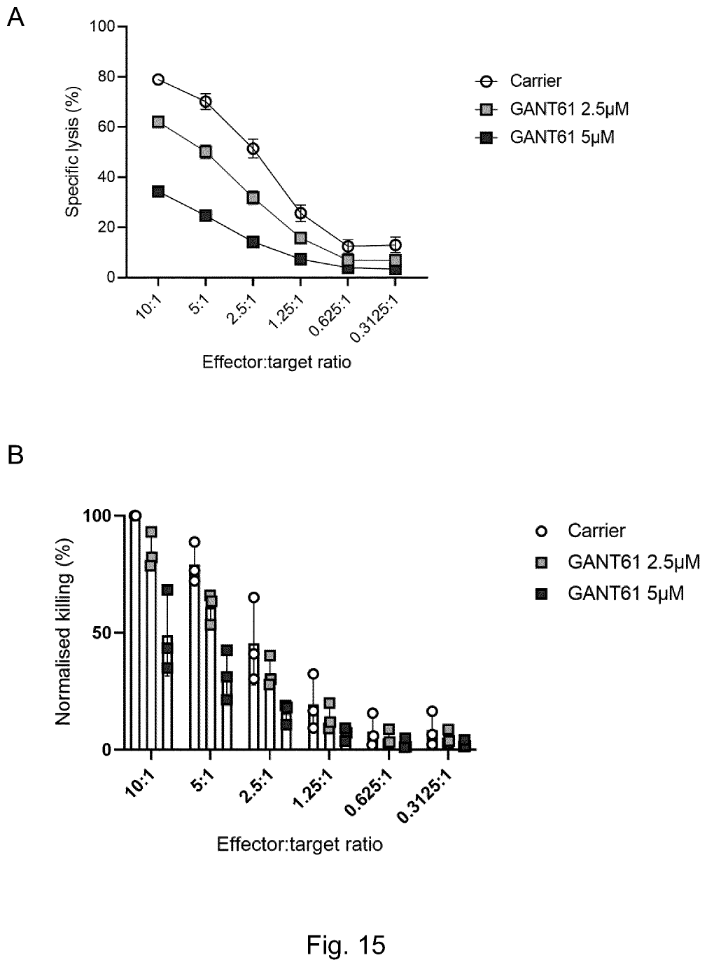

It is also known that Hh signalling in murine CD8+ T cells is not inducible via administration of extracellular Hh ligands (de la Roche et al. (2013)). Instead, Hh induction occurs via the modulation of L-type voltage-gated calcium (Cav) channels downstream of the T cell receptor. For example, Hanna et al. (2021) report that nifedipine-sensitive Cavs control Gli1 induction in naïve CD8+ T cells and CD8+ CTLs following TCR stimulation. It is also reported that Cav1 family channels mediate specific cell killing by murine CD8+ CTLs in a Gli1-dependent manner, functioning independently of canonical Hh signalling. The authors further demonstrate that Cav1 family channels are responsible for Gli1 induction in human CTLs. As shown in FIG. 15, the GLI1 inhibitor GANT61 reduces tumour cell killing of human CD8+ cytotoxic lymphocytes, in line with the known role of Gli1 in tumour cell killing.

However, it is widely recognised that calcium signalling processes are both highly complex and tightly regulated. The role of L-type Cav channel agonists in controlling lymphocyte activation and effector function is poorly understood.

### Experiment 1: L-Type Cav Agonists Specifically Enhance Gli1 Transcript Levels in TCR-Stimulated Murine CD8+ CTLs and Potentiate Specific Tumour Cell Killing

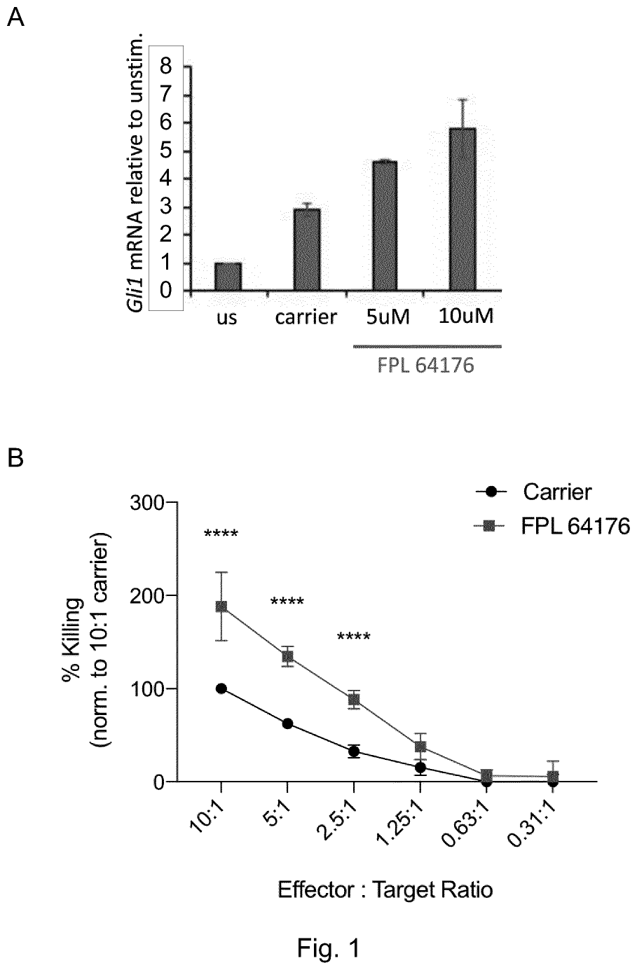

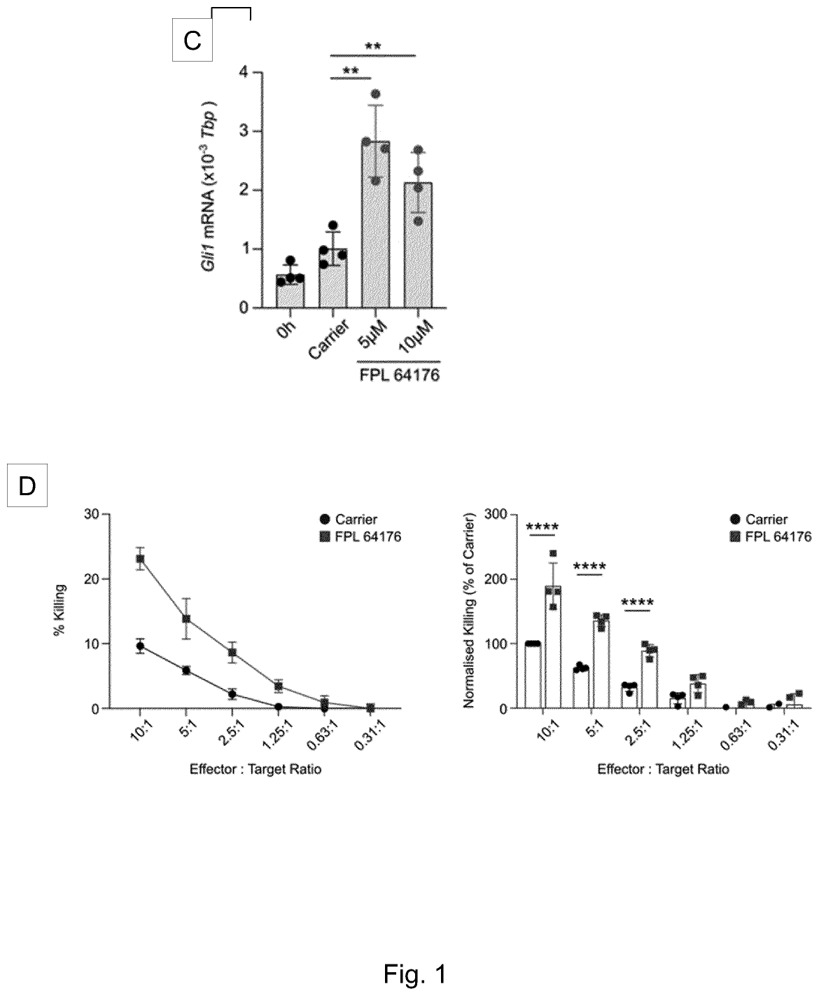

To investigate a potential role for L-type Cav channel agonists in modulating CD8+ CTL activation and effector function, CD8+ T cells were isolated from the spleen and inguinal lymph nodes of Rag2−/− OT-I mice and CTLs were generated. For reference, Rag2−/− mice do not develop mature T and B lymphocytes. OT-I mice bear a transgene encoding for a TCR that recognises a chicken ovalbumin (Ova) peptide fragment. Rag2−/− OT-I mice facilitate the isolation of naïve CD8+ T cell populations. Plates were coated using 2.5 μg/mL anti-CD3ε mAb and CD8+ CTLs were stimulated for 3 hours via the TCR in the presence of 5 or 10 μM FPL 64176, an L-type Cav channel agonist, or carrier control. Note that CD8+ CTLs were pre-incubated with FPL 64176 or carrier control for 40-50 mins before stimulation. Gli1 expression was assessed using qRT-PCR. As demonstrated in FIGS. 1A and 1C, FPL 64176 administration resulted in a dose-dependent increase in Gli1 expression in murine CD8+ CTLs.

In order to establish whether this increase in Gli1 expression was correlated with an increase in cell effector function, an LDH cytotoxicity assay was performed. LDH (lactate dehydrogenase) is a stable cytoplasmic enzyme, that is released into the extracellular environment following damage to the plasma membrane. The LDH assay assesses cell viability by way of measuring LDH enzymatic activity in cell culture supernatant. In brief, CD8+ CTLs derived from Rag2−/− OT-I mice were co-cultured with Ova peptide-pulsed EL-4 target cells at the effector:target ratios indicated in FIGS. 1B and 1D, in the presence of 10 μM FPL 64176 or carrier control. As shown, FPL 64176 administration improved the specific cell killing of EL-4 tumour cells by at least 100% over a range or effector-to-target ratios, suggesting that an increase in the expression of Gli1 acts to enhance the effector function of CD8+ CTLs.

To evaluate whether other clinically-approved drugs with potential L-type Cav channel agonist activity could enhance Gli1 transcription in addition to FPL 64176, CTLs were stimulated in the presence of FPL 64176, (R)-Baclofen and Nefiracetam, and Gli1 transcription was assessed using qRT-PCR. For reference, whilst FPL 64176 acts as a potent selective activator of L-type Ca²⁺ channels, (R)-Baclofen acts as selective GABA_{B} receptor agonist and has been described to facilitate L-type channel currents in some settings, and Nefiracetam acts as a presumed GABA_{A} receptor agonist with some L-type voltage-gated calcium channel agonistic activity.

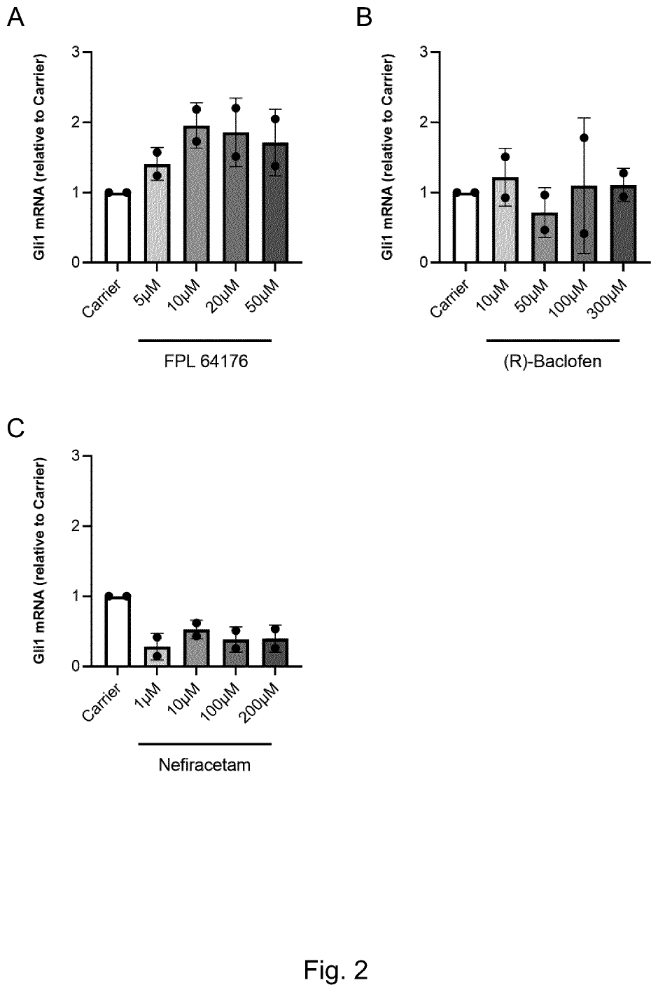

In brief, naïve CD8+ T cells were isolated from the spleen and inguinal lymph nodes of C57BL/6 wildtype mice and CTLs were generated. CTLs were restimulated on plates precoated with anti-CD3ε (2.5 μg/ml) antibody for 6 hours in the presence of indicated concentrations of FPL 64176; (FIG. 2A); (R)-Baclofen (FIG. 2B); Nefiracetam (FIG. 2C) (or respective carrier controls). Note that CD8+ CTLs were also pre-incubated with the appropriate agent or carrier control for 40-50 mins prior to restimulation. RNA was extracted from cells and Gli1 expression was assessed by qRT-PCR. As shown in FIG. 2, only FPL 64176 successfully enhances Gli1 expression following TCR activation. In contrast, (R)-Baclofen administration had little effect on Gli1 expression, and Nefiracetam reduced Gli1 expression by around 50%. These data indicate that potent and specific agonists of L-type Cav channels can increase Gli1 expression in murine CTLs.

### Experiment 2—L-Type Cav Agonists Enhance GLI1 Expression in TCR-Stimulated Naive CD8+ T Cells and CTLs and Potentiate Specific Tumour Cell Killing

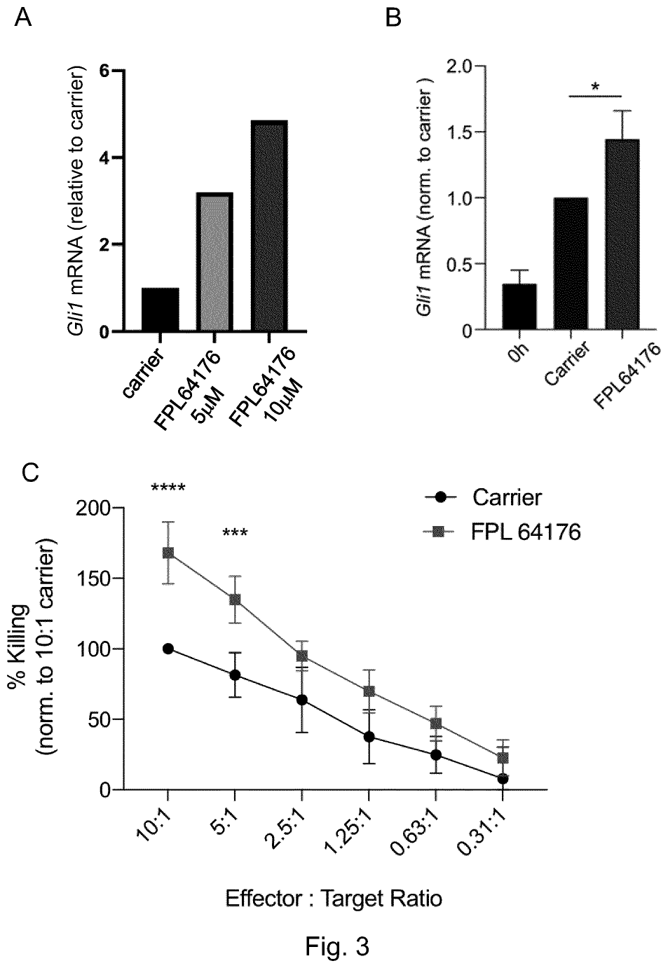

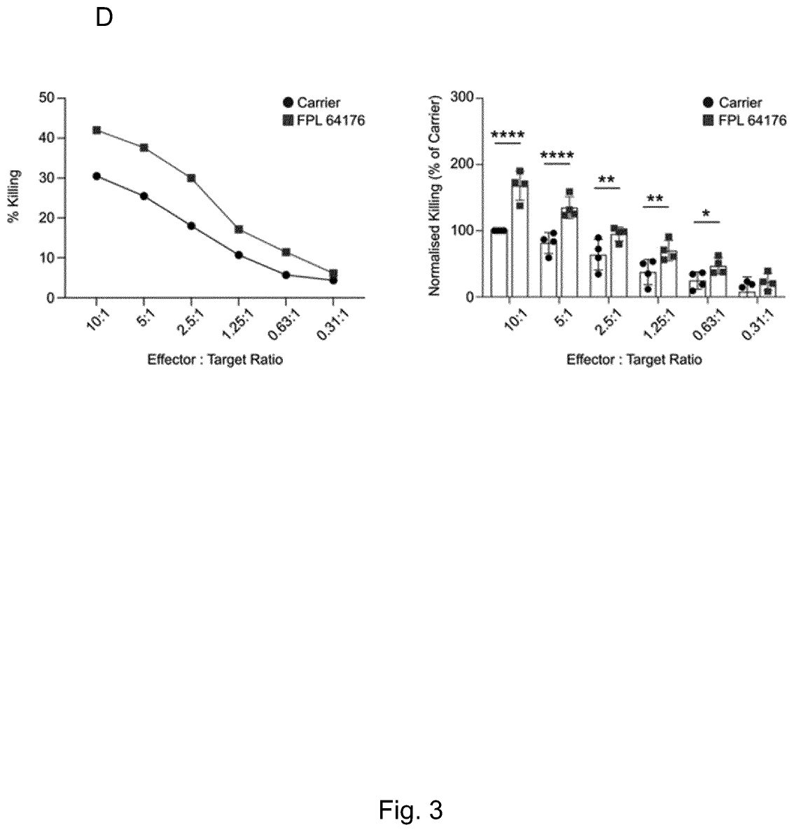

In order to assess the role of L-type Cav channel agonists in controlling the activation and effector function of human CD8+ T cells, naive primary human CD8+ T cells were purified from the buffy coat layer of blood isolated from healthy donors. Cells were stimulated using ImmunoCult™ Human CD3/CD28/CD2 T Cell Activator beads for 24 hours in the presence of 5 or 10 μM FPL 64176 or carrier control, and GLI1 mRNA expression was assessed by qRT-PCR. As shown in FIG. 3A, FPL 64176 administration led to a dose-dependent increase in GLI1 expression. Next, human CD8+ CTLs were restimulated between day 15 and day 20 post initial TCR stimulation with plate-bound anti-CD3 in the presence of 10 μM FPL 64176 or carrier control for 24 hours, and GLI1 mRNA expression was assessed by qRT-PCR. As shown in FIG. 3B, FPL 64176 administration led to an increase in GLI1 expression. Together, data from FIGS. 3A and 3B corroborate data generated using murine CD8+ CTLs, as provided in Experiment 1.

A VITAL assay was subsequently performed to assess the role of L-type Cav agonists with regards to specific tumour cell killing by human CD8+ CTLs. The VITAL assay is a flow cytometry based assay used widely for the assessment of cell killing. It was hypothesised that an increase in GLI1 expression would lead to an enhancement in specific tumour cell killing. In brief, CTLs were co-cultured with CFSE-labelled P815 target cells at the indicated effector to target cell ratios in the presence of FPL 64176 or carrier control (FIGS. 3C & 3D). Note that CD8+ CTLs were also pre-incubated with the appropriate agent or carrier control for 40-50 mins prior to the VITAL assay. As shown, FPL 64176 administration improved cell killing by ˜70%, therefore supporting the hypothesis that increase of GLI1 expression downstream of L-type Cav channel activation acts to enhance the effector function of CD8+ T cells. For clarity, this observation corroborates the data generated using murine CD8+ CTLs, as provided in Experiment 1.

### Experiment 3—Gill Expression is Critical for the Cytolytic Function of Murine NK Cells

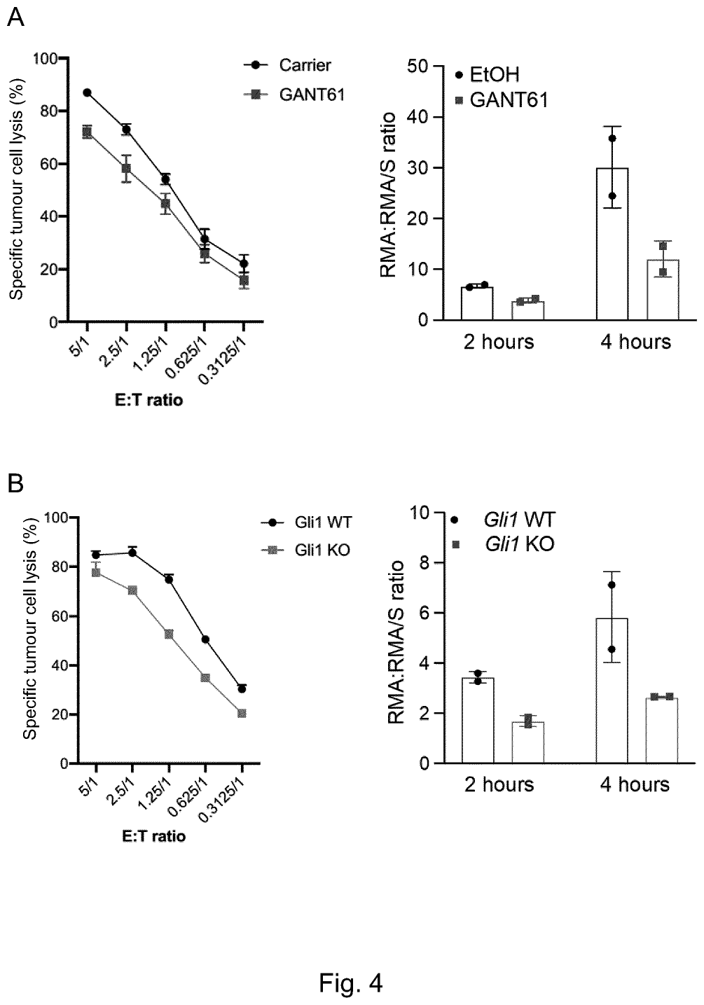

It was hypothesised that the therapeutic effect of L-type Cav channel agonists may be conserved between CD8+ T and NK cells. To investigate this hypothesis, NK cells were isolated from spleens of C57BL/6 wildtype mice using a MACS negative isolation protocol, and expanded in vitro for 7 days in the presence of 500 IU/L IL-2. An LDH assay (FIG. 4, left panels) and VITAL assay (FIG. 4, right panels, RMA tumour target cells are insensitive to NK cell killing and were used as a reference in the VITAL assay) were performed to assess specific tumour cell killing. In brief, NK cells were co-cultured with NK-sensitive RMA/S cells, in the presence of 5 μM GANT61 or carrier control. For reference, GANT61 (NSC 136476) inhibits binding of both Gli1 and Gli2 to DNA. As shown in FIG. 4A, NK cells administered GANT61 were found to exhibit a ˜15% decrease in cell killing, as measured by the LDH assay, compared to NK cells treated using the carrier control. Similarly, specific tumour cell killing, as determined in the VITAL assay (FIG. 4A, right hand panel) was greatly reduced in cells treated with GANT61 as compared to carrier control.

In order to elucidate the contribution of Gli1 to NK cell killing more specifically, NK cells were isolated from WT and Gli1−/− (KO) mice. Cells were co-cultured with RMA/S cells as described above (without GANT61 treatment), and cell killing was assessed using both an LDH assay (left panel) and a VITAL assay (right panel, with NK-insensitive RMA cells as controls). As shown in FIG. 4B, Gli1 KO NK cells were found to exhibit a reduction in cell killing that was similar in magnitude to that exhibited by GANT61-treated NK cells (FIG. 4A). In combination, these data demonstrate that Gli1 plays an important role in mediating the cytolytic function of NK cells, thereby corroborating the data provided in Experiments 1 and 2.

### Experiment 4—L-Type Cav Agonists Drive Gli1 Transcription and Promote Specific Cell Killing by Human NK Cells

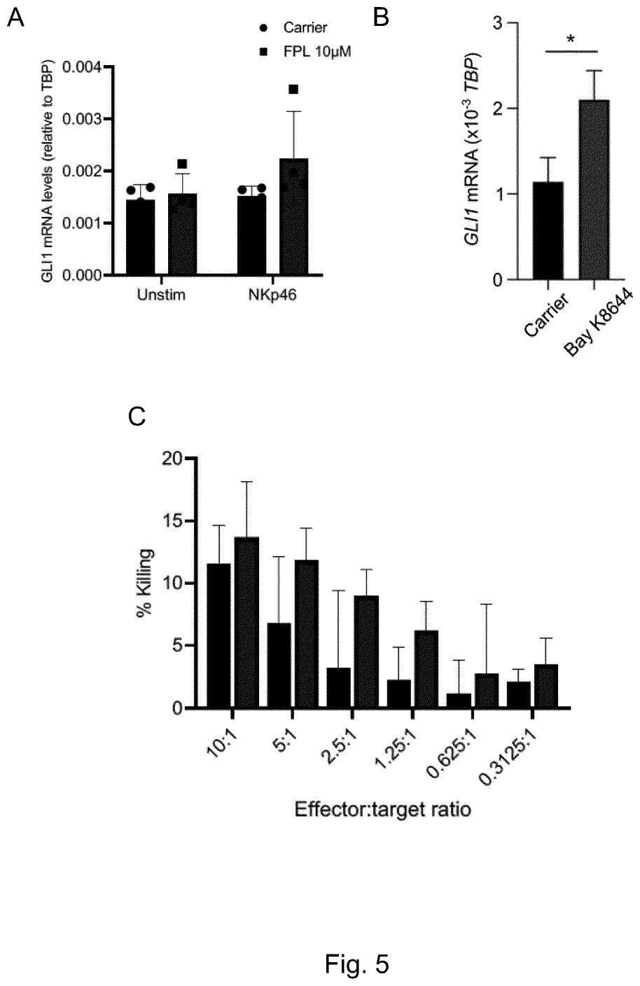

To investigate whether L-type Cav channel agonists are also capable of enhancing GLI1 mRNA levels in the human NK cell line NK-92 (a non-Hodgkin's lymphoma cell line, expressing only Cav1.3), NK-92 cells were stimulated using an anti-NKp46 mAb (5 μg/mL) for 4 hours in the presence of either 10 μM FPL 64176 or 100 μM Bay K8644. For reference, Bay K8644 is a structural analogue of nifedipine, and acts as an L-type Cav channel agonist. GLI1 expression was assessed using qRT-PCR. As shown in FIG. 5A, GLI1 expression was increased in NK-92 cells stimulated using anti-NKp46 mAb in the presence of FPL 64176, as compared to cells stimulated in the presence of carrier control. No significant increase was observed in unstimulated cells in the presence of FPL 64176. Similarly, GLI1 mRNA levels were increased (almost two-fold) in NK-92 cells stimulated using Bay K8644, as compared to cells treated with carrier control (as per FIG. 5B). These observations confirm that the GLI1-inducing effect of Cav agonists is not merely restricted to the compound FPL 64176. In particular, in NK cells.

In order to establish whether an increase in GLI1 expression is correlated with an increase in NK-92 cell effector function, NK-92 were co-cultured with K562 myelogenous leukaemia cells in the presence of 10 μM FPL 64176 or carrier control, at the effector:target ratios indicated in FIG. 5C. Cell killing was assessed using the LDH assay, as previously described. As shown in FIG. 5C, FPL 64176 administration enhanced target cell killing by NK-92 cells, as compared to cells administered the carrier control.

In combination, these data corroborate the data generated in murine and human CD8+ CTLs, as provided in Experiments 1 and 2, thereby confirming that the mechanism of action of L-type Cav agonists (for example including FPL 64176 and Bay K8644) is conserved within cytotoxic lymphocyte populations. In particular, these data demonstrate that the effect of L-type Cav channel agonists is at least conserved between murine and human CD8+ CTLs and NK cells.

### Experiment 5—Mutations Driving a Gain-of-Function Hh Phenotype Act to Potentiate Specific Cell Killing by NK Cells

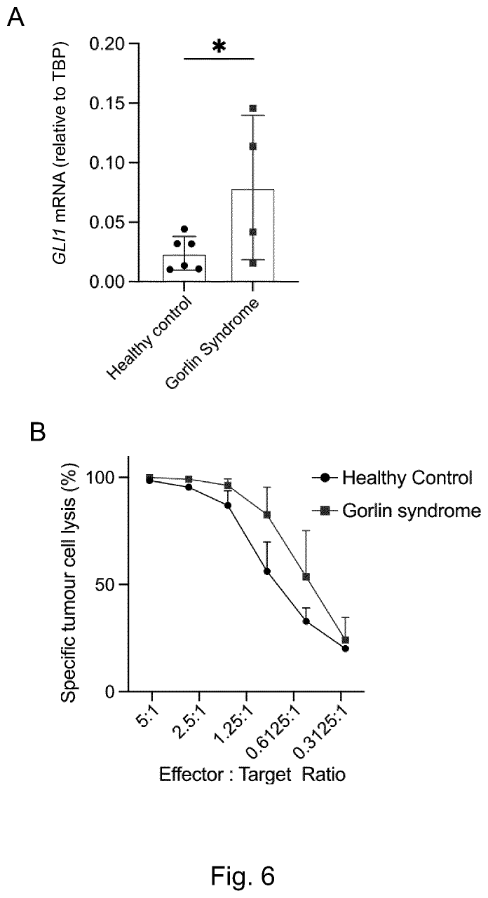

Gorlin syndrome patients are known to carry one of a number of possible germline mutations and deletions in the protein patched homolog 1 (PTCH1) gene PTCH1, which cause a loss-of-function of PTCH1. PTCH1 is a negative regulator of the pathway. Therefore, PTCH1 loss-of-function mutations enhance canonical Hh signalling and enhance GLI1. In order to assess the effect of PTCH1 mutations, NK cells were isolated from Gorlin syndrome patients and healthy control individuals, and cultured for two days in IL-2. On day 2 levels of GLI1 mRNA were determined by qRT-PCR (FIG. 6A). As shown NK cells from Gorlin patients have higher GLI1 mRNA levels. Next, NK cells from Gorlin syndrome patients and healthy control individuals were co-cultured with K562 target cells at the effector to target ratios indicated in FIG. 6B. As shown, NK cells isolated from patients with Gorlin syndrome were observed to act as more potent killers than NK cells isolated from healthy control individuals. Without wishing to be bound by any particular theory, the present inventors believe that approaches aimed at suppressing, reducing or down-regulating PTCH1 function in immune cells—and the associated increase is GLI1 signalling—will recapitulate the enhanced cell killing observed by the NK cells isolated from patients with Gorlin syndrome. Accordingly, immune cells engineered to decrease PTCH1 function and/or expression or gene silencing approaches such as CRISPR or siRNA-based silencing of PTCH1 in immune cells may provide new immune cell-based therapeutic avenues for the treatment of various cancers.

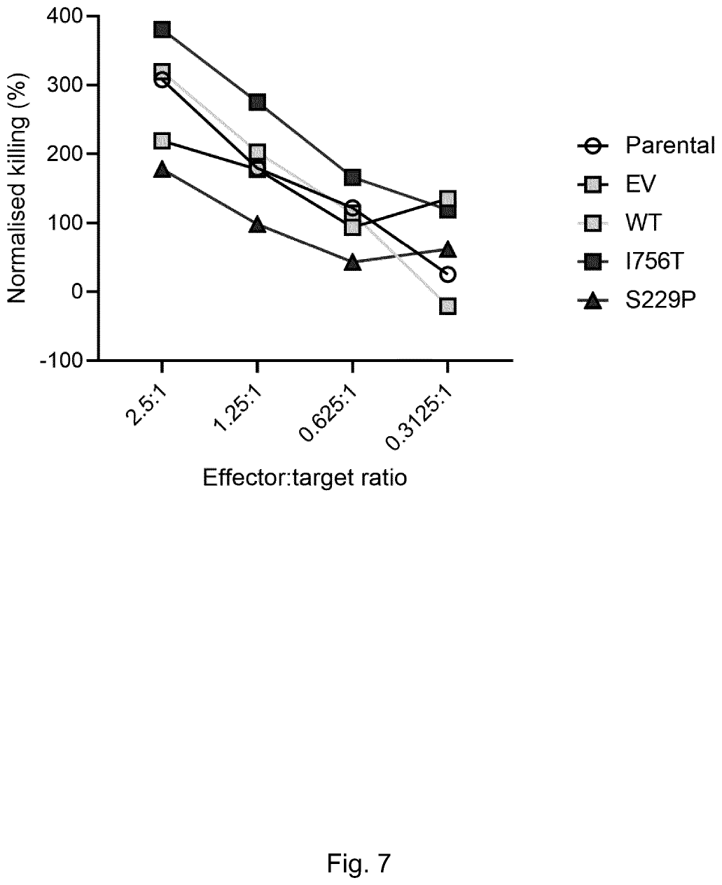

To determine if the increased specific cell killing seen in Gorlin patients can be achieved by engineering cells to express a gain-of-function mutation resulting in increased Gli1 signalling, NK-92 cells were engineered to stably express the I756T GOF mutation in Cav1.4. Empty vector (EV) and Cav1.4 (WT) were used as controls. The loss-of-function mutation S229P was used as a negative control. Killing of K562 tumour cells was assessed after 24 h by LDH killing assay. As shown in FIG. 7, the I756T mutant led to increased cell killing. Therefore, the gain-of-function effect in the non canonical Hedgehog pathway can be achieved through genetic engineering of Cav channels. These mutations do not affect cell survival and proliferation (data not shown).

### Experiment 6—Gill Transcription Mediates Specific Cell Killing by Murine Cytotoxic CD4+ T Cells

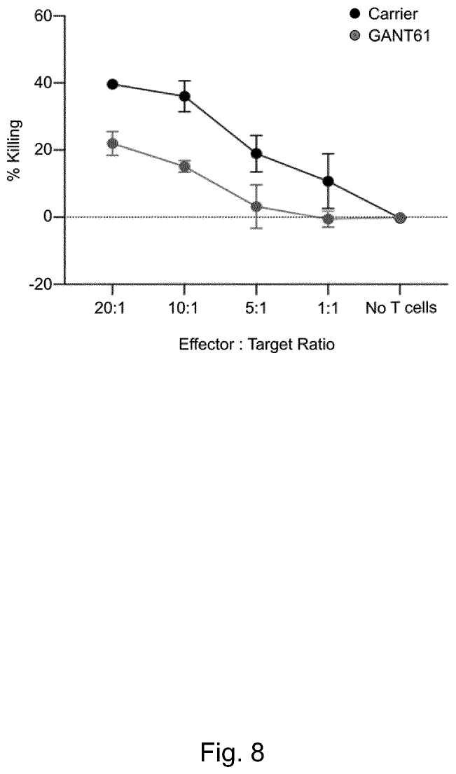

Experiments 1, 2, 3 and 4 identify Gli1 transcription as a mediator of specific cell killing in CD8+ CTLs and in NK cells, in humans and in mice. To investigate whether Gli1 also plays a role in driving the effector function of CD4+ T cells, cells were isolated from the spleens of OT-II mice and CTLs were generated. Cells were treated using GANT61 at a concentration of 5 μM or carrier control, 24 h prior to being co-cultured with B cells (as target cells) at the effector:target ratios indicated in FIG. 8. As shown, CD4+ CTLs administered GANT61 exhibited a 50% reduction in specific cell killing as compared to CD4+ CTLs administered carrier control. This observation suggests that the function of Gli1 in promoting cellular effector function is conserved between CD8+ and CD4+ CTLs, and NK cells, thereby corroborating the results of Experiments 1, 2, 3 and 4.

### Experiment 7—Cytokine Production and Cytolytic Effector Maintenance are Unaffected by Gli1 Inhibition in all Cytotoxic Lymphocytes

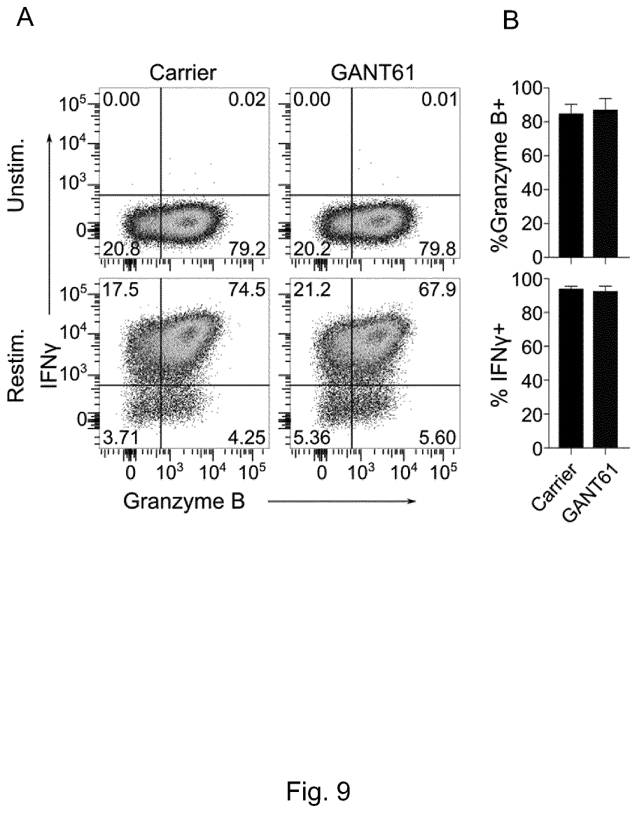

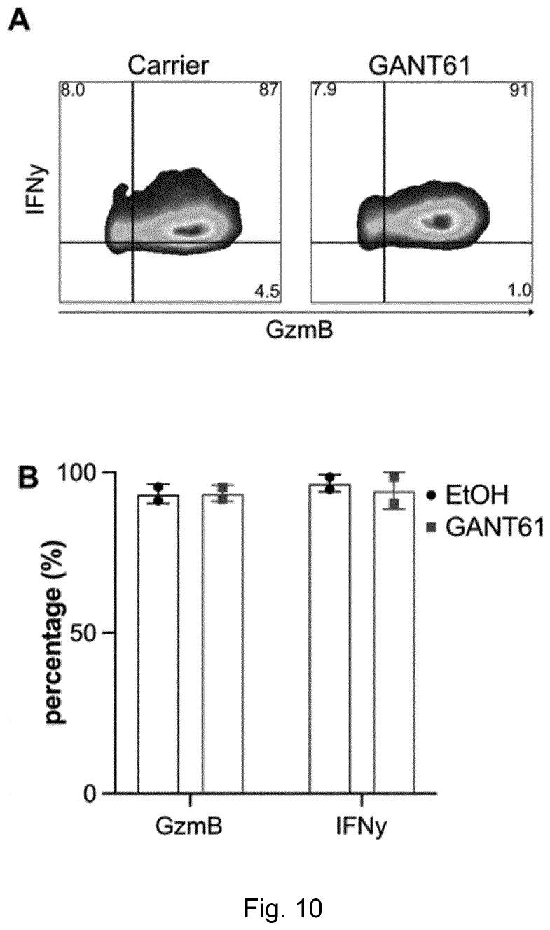

The data shown in Examples 1-6 demonstrate a novel TCR-induced, L-type Cav channel-mediated regulation of killing via Gli1. To investigate this further, the effect of GANT61 inhibition on Granzyme B and IFNγ secretion was investigated. Cytotoxic CD4+ cells were generated from OT-II mice and treated with 5 μM GANT61 or carrier control on day 7 for 24 h prior to flow cytometric analysis of Granzyme B (GzmB) and IFNγ levels on day 8. NK cells were isolated from C57BL/6 mice and treated with 10 μM GANT61 or carrier control (EtOH) overnight (˜18 hours) prior to analysis of intracellular granzyme B (GzmB) and IFNγ expression by flow cytometry. In both cytotoxic CD4+ cells (FIG. 9) and NK cells (FIG. 10), the GzmB content and IFNγ secretion were unaffected by Gli inhibition.

FIGS. 9 and 10 show that the pathway uncovered by the inventors behaves differently to reported roles of Ca2+ channels on cytokine production and cytolytic effector maintenance. Interestingly, the inventors have previously shown the same effect in cytotoxic CD8+ T cells (De la Roche, 2013), indicating that this mechanism is shared between all cytotoxic lymphocytes.

### Experiment 8—L-Type Cav Agonist Treatment does not Impair CD8⁺ T Cell Viability or Proliferation, does not Affect Memory Phenotypes, and Enhances CTL Killing Via Extracellular Ca²⁺

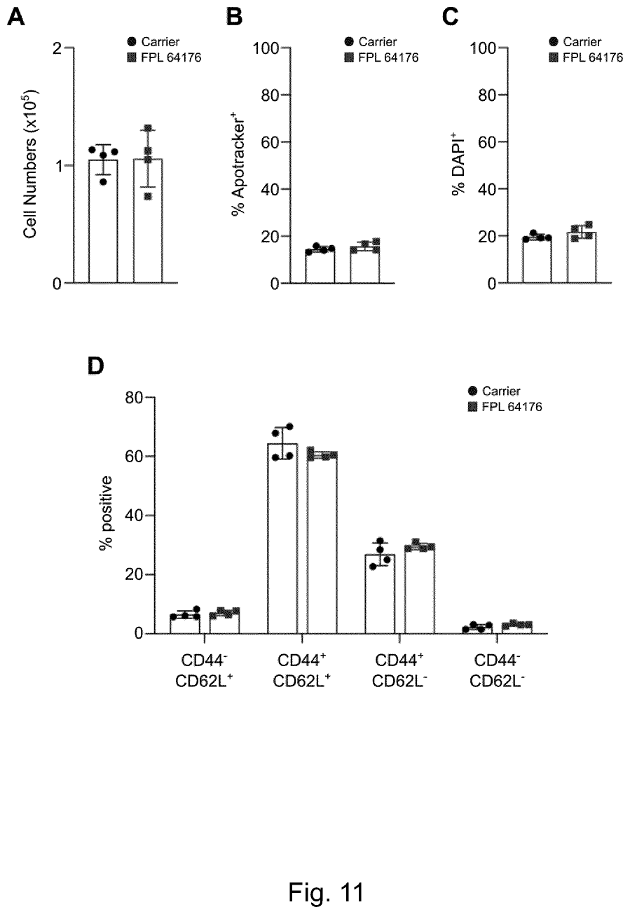

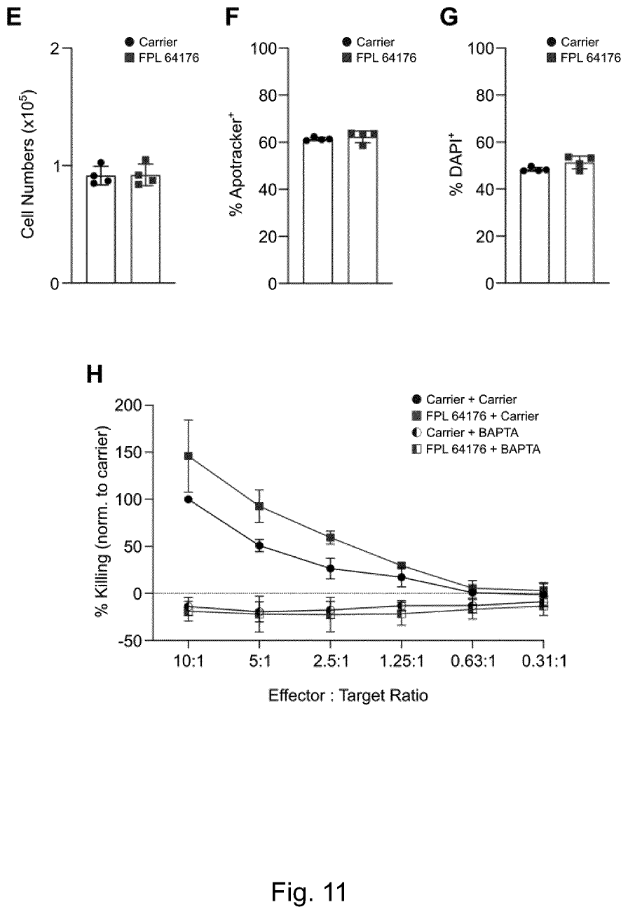

To ensure that the use of L-type Cav agonist is not adversely affecting immune cells, general cell functions were assessed. Naïve CD8⁺ T cells were stimulated with plate-bound anti-CD3/CD28 antibodies for 24 h in the presence of 10 μM FPL64176 or carrier control before flow cytometric analysis (FIG. 11A-D). The L-type Cav agonist did not affect CD8⁺ T cell viability or proliferation, as shown by analysis of live cell numbers (A), analysis of cells positive for the apoptotic marker Apotracker™ Green (B), and analysis of cells positive for cell viability dye DAPI (C). Central and effector memory was also shown to be unaffected (D), with no significant change between agonist and control following analysis of T cell subsets based on the expression of CD44 and CD62L: CD44⁻ CD62L⁺ (naïve), CD44⁺ CD62L⁺ (central memory), and CD44⁺ CD62L⁻ (effector memory). The viability and proliferation was maintained following restimulation of the CTLs on day 10 with plate-bound anti-CD3 antibody for 24 h in the presence of 10 μM FPL64176 or carrier control before flow cytometric analysis (E-G). To determine whether the Gli1-mediated tumour cell killing requires extracellular Ca²⁺, the calcium chelator BAPTA was added to an LDH cytotoxicity assay. BAPTA will sequester any extracellular Ca²⁺ and thus prevent immune cell killing if the Gli1 pathway requires extracellular calcium. As can be seen in FIG. 11H, immune cell killing was depleted in the presence of BAPTA. Therefore, Gli1-mediated killing relies on the presence of extracellular Ca²⁺.

### Experiment 9—L-Type Cav Agonists Increase the Killing Capacity of Human Gammadelta T Cells

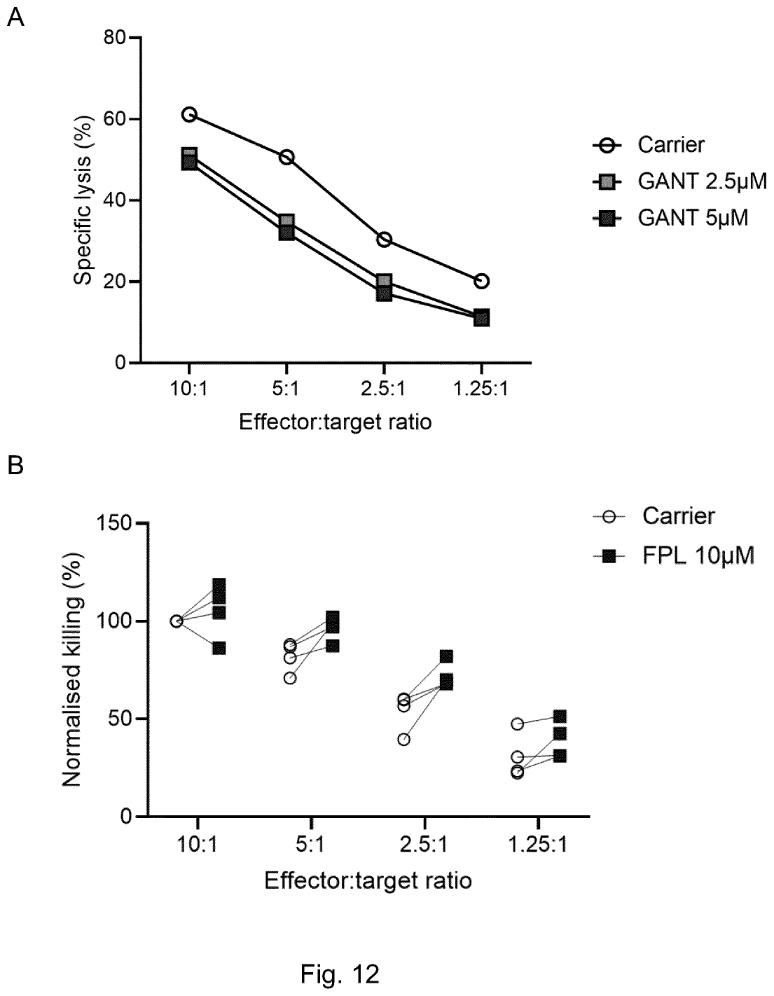

Experiments 1-6 show that GLI1 transcription is a mediator of specific cell killing in CD8⁺ CTLs and in NK cells in both humans and mice, and also plays a role in driving the effector function of CD4⁺ T cells in mice. To investigate whether GLI1 also plays a role in driving the effector function of gammadelta T cells, Vy9Vd2 cells were prepared from PBMCs of healthy human donors. Killing assays were conducted between day 16-20. FIG. 12A shows reduced killing capacity of K562 target cells by gammadelta T cells pre-treated with GLI1 inhibitor GANT61. Gammadelta T cells were also pre-treated with 10 μM FPL 64176 or ETOH (carrier control) before being co-cultured with K562 target cells at indicated effector to target ratios and subjected to a flow-cytometry cytotoxicity assay. FIG. 12B shows an increased killing in gammadelta T cells treated with FPL 64176 compared to control. Therefore, Gli1 also controls the killing ability of gammadelta T cells and killing of these cells can be enhanced by L-type Cav agonists.

### Experiment 10—Treatment of Tumour-Specific CD8 T Cells with Cav Channel Agonist FPL 64176 Before Adoptive Transfer into Tumour-Bearing Mice Leads to Increased Tumour Cell Death In Vivo

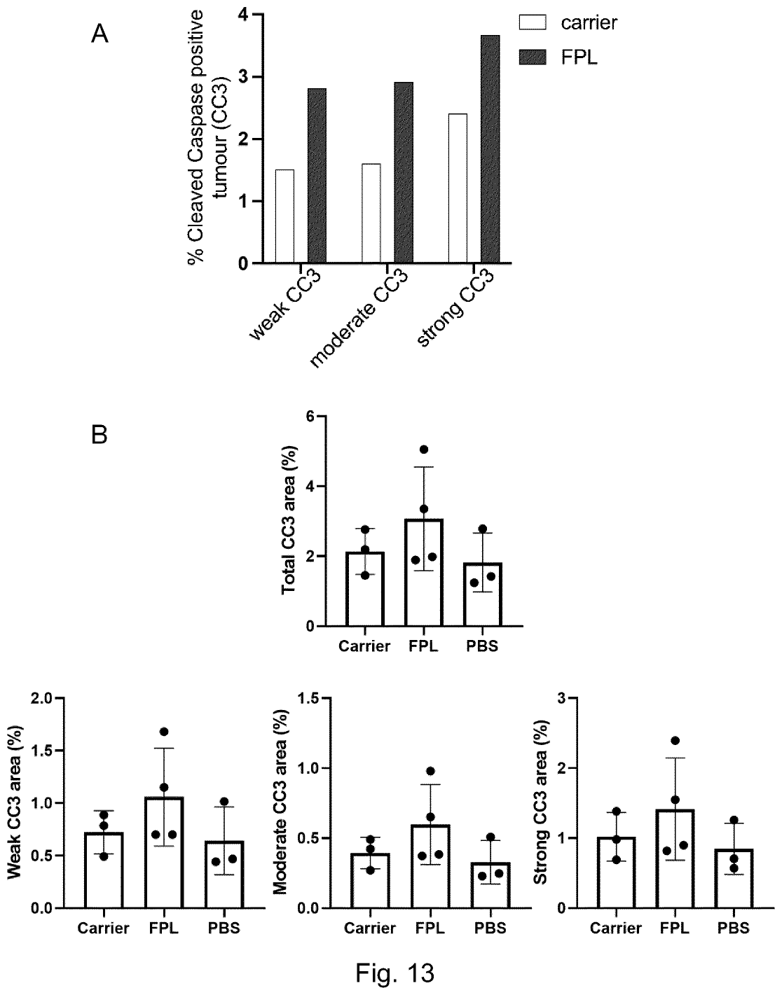

As described above, the present invention may find use in adoptive transfer. To investigate the applicability of Cav channel agonist treatment in an adoptive transfer treatment regime, cytotoxic OTI T cells were re-stimulated in vitro in the presence of FPL 64176 or carrier control before adoptive transfer into MC38-Ova tumour bearing Rag2 KO mice via tail vein injection. 24 hours after the adoptive transfer, tumours were excised and analysed by immunohistochemistry for Cleaved Caspase 3. FIGS. 13A&B indicate that treatment of tumour-specific T cells with a Cav channel agonist prior to adoptive transfer leads to increased tumour cell killing compared to adoptive transfer with T cells that have not been treated with a Cav channel agonist. This data establishes the feasibility of the present invention for use in adoptive transfer. FPL treatment may not affect effector and central memory phenotypes in tumour, spleen and draining lymph node in vivo (data not shown).

### Experiment 11—Impact of L-Type Voltage Gated Ca²⁺ Channel Agonist FPL 64176 on Ca²⁺ Flux

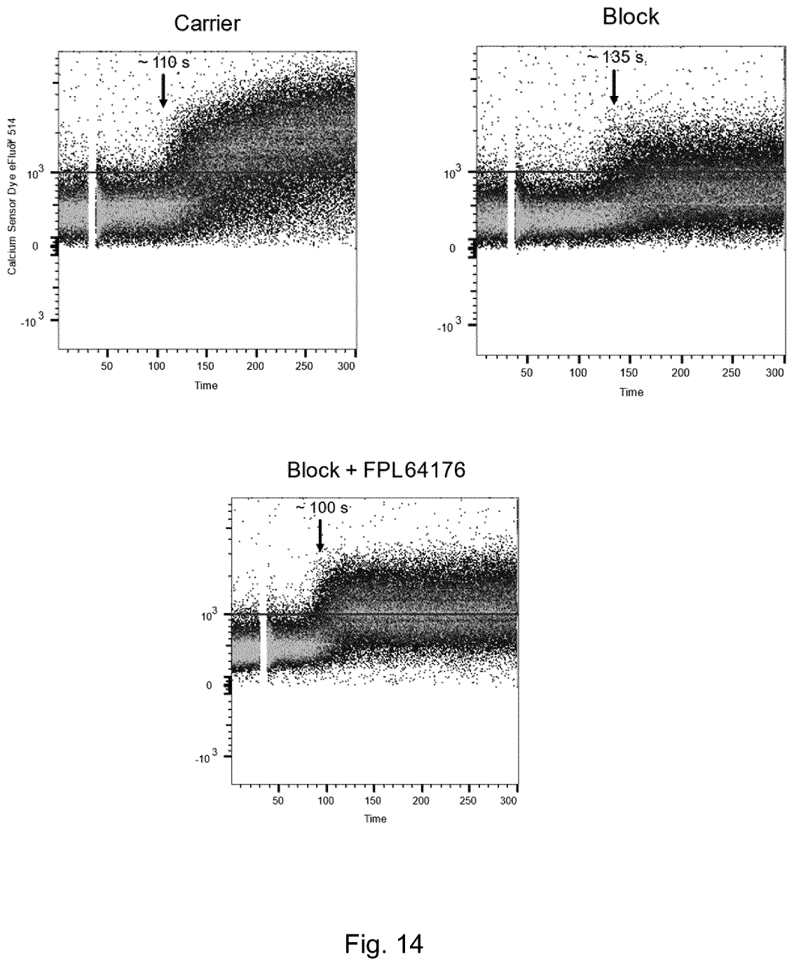

To investigate the mechanism by which L-type Cav agonists exert their effect, calcium flux experiments were performed on murine CD8⁺ cytotoxic T lymphocytes. FIG. 14 demonstrates that treatment with the L-type voltage gated Ca²⁺ channel agonist FPL 64176 accelerates the initiation of the Ca²⁺ flux following TCR crosslinking and specifically increases Cav-mediated Ca²⁺ flux in murine CD8⁺ cytotoxic T lymphocytes.

### SUMMARY

Cytotoxic lymphocytes, in particular CD8+ T cells and NK cells, are critical for the elimination of infected and malignant cells. With the unprecedented successes of immunotherapies harnessing the killing potential of cytotoxic lymphocytes, ways of enhancing their killing potential are being explored. Advantages of the present invention may be seen when comparing the results disclosed herein to other approaches.

One example described in the art is the deletion of the transcription factor Bach2 in CD8+ T cells, described in Barton (2022). Bach2 is highly expressed in naïve T cells and prevents terminal differentiation to effector cells by limiting the expression of TCR-driven genes and by increasing the generation of memory cells. Furthermore, Bach2 is critical for the differentiation of stem-like CD8+ T cells that are essential for tumour control and response to checkpoint blockade in vivo.

Barton (2022) shows that genetic knockout of Bach2 in CD8+ T cells led to 50-75% enhanced murine CTL killing in vitro in bulk populations (FIG. 1 of Barton 2022). In contrast, the present invention displays a 100% increase in murine CTL killing (Example 1 and FIG. 1). Moreover, in Barton (2022), the killing phenotype was in part due to the altered differentiation phenotype in Bach2^{-/-} CD8+ T cells, the differential proportion of CD8 T cells in the tested population and enhanced proliferation of the Bach2^{-/-} T cells. When CTLs were obtained from similar starting populations, CTL killing was only enhanced 15-50% and due to enlarged cytolytic granules containing higher levels of lytic proteins.

The present invention leads to greater overall killing and does not affect proliferation, differentiation and overall levels of lytic proteins. This is in contrast to the Bach2^{-/-} T cells of Barton (2022), which are expected to terminally differentiate in vivo. The present invention specifically targets the killing mechanism.

The present invention utilises a novel non-canonical Hedgehog signalling pathway downstream of the TCR that is independent from NFAT, AP-1, and NFkB signalling and associated calcium fluxes (Hanna, 2021). Importantly, NFAT has been shown to be a critical driver of exhaustion in T cells (Martinez, 2015). As such, it is expected that the present invention will not induce an exhausted phenotype.

The data provided in Experiments 1, 2, 3 and 4 demonstrate that L-type Cav agonists act to enhance expression of Gli1 in murine and human CD8+ CTLs and NK cells; and act to potentiate the specific cell killing of multiple tumour cell types. The data provided in Experiment 7 indicate that this effect further extends to CD4+ CTLs, and Experiment 9 shows the effect also extends to gammadelta T cells. These are the first experimental data which link L-type Cav channel agonists (such as FPL 64176 and Bay K8644) to Hedgehog activation and Gli1 transcription, and which utilise L-type Cav channel agonists to potentiate the cytotoxic effector function of CD8+ CTLs, NK cells and CD4+ CTLs. Experiment 10 shows this approach may find use in adoptive transfer.

In summary, the experimental data provided act to affirm the efficacy and therapeutic utility of the invention, demonstrating that L-type Cav agonists and/or immune cells expressing engineered L-type Cav channels are likely to be valuable tools for use in the treatment of disease.

### REFERENCES

A number of publications are cited above in order to more fully describe and disclose the invention and the state of the art to which the invention pertains. Full citations for these references are provided below. The entirety of each of these references is incorporated herein by reference.
Anzalone et al., Search-and-replace genome editing without double-strand breaks or donor DNA. Nature (2019) 576:149-157
Badou et al., Emerging roles of L-type voltage-gated and other calcium channels in T lymphocytes. Front Immunol 4, 243 (2013).

Barton, P. R. et al. Super-killer CTLs are generated by single gene deletion of Bach2. Eur J Immunol 52, 1776-1788 (2022) de la Roche et al., Hedgehog Signaling Controls T Cell Killing at the Immunological Synapse. Science (2013)
Hamara-Wahanui et al., A CACNA1F mutation identified in an X-linked retinal disorder shifts the voltage dependence of Cav1.4 channel activation. PNAS (2005) 102(21)
Hanna et al., Non-canonical Hedgehog signaling through L-type voltage gated Ca²⁺ channels controls CD8⁺ T cell killing. BioR_{X}iv (2021)
Martinez, G. J. et al. The transcription factor NFAT promotes exhaustion of activated CD8(+) T cells. Immunity 42, 265-278 (2015).
Kotturi and Jefferies, Molecular characterization of L-type calcium channel splice variants expressed in human T lymphocytes. Molecular Immunology (2005)
Striessnig, et al. Voltage-Gated Ca²⁺-Channel al-Subunit de novo Missense Mutations: Gain or Loss of Function—Implications for Potential Therapies. Frontiers in Synaptic Neuroscience (2021) 13(634760)
Williams et al., Functional impact of a congenital stationary night blindeness type 2 mutation depends on subunit composition of Cav1.4 Ca(2+) channels. J Biol. Chem. (2020) 295:17215-17226
For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. An engineered immune cell, wherein the engineered immune cell comprises a gain-of-function mutation in a Cav channel and/or overexpresses an L-type voltage-gated calcium (Cav) channel.

2. The engineered immune cell of claim 1, wherein the engineered immune cell comprises a gain-of-function mutation in CACNA1F.

3. The engineered immune cell of claim 1 or claim 2, wherein the engineered immune cell overexpresses Cav1.4 in comparison with a wild-type immune cell of the same type.

4. The engineered immune cell of any preceding claim, wherein the CACNA1F mutation comprises I745T.

5. The engineered immune cell of any preceding claim, wherein the engineered immune cell is selected from the group consisting of a T cell, a CAR-T cell, an NK cell and a CAR-NK cell.

6. An engineered immune cell for use in medicine, wherein the engineered immune cell comprises a gain-of-function mutation in an L-type Cav channel and/or overexpresses an L-type Cav channel.

7. An engineered immune cell for use in a method of treating cancer in a subject, wherein the engineered immune cell comprises a gain-of-function mutation in an L-type Cav channel and/or overexpresses an L-type Cav channel.

8. The engineered immune cell for the use according to claim 7, wherein the cancer is selected from melanoma, lung cancer, colorectal cancer, breast cancer, lymphoma, leukaemia, multiple myeloma, liver cancer, stomach cancer, central nervous system (CNS) tumour, paediatric glioma, ovarian cancer, cervical cancer, colon cancer, pancreatic cancer, prostate cancer, and gastric cancer.

9. The engineered immune cell for use according to any one of claims 6 to 8, wherein the engineered immune cell comprises a gain-of-function mutation in CACNA1F.

10. The engineered immune cell for use according to any one of claims 6 to 9, wherein the engineered immune cell overexpresses Cav1.4 in comparison with a wild-type immune cell of the same type.

11. The engineered immune cell for use according to any one of claims 6 to 10, wherein the CACNA1F mutation comprises I745T.

12. The engineered immune cell for use according to any one of claims 6 to 11, wherein the engineered immune cell is selected from the group consisting of a T cell, a CAR-T cell, an NK cell and a CAR-NK cell.

13. An L-type Cav channel agonist for use in a method of stimulating cell killing activity of an immune cell in a subject having a proliferative disorder.

14. A pharmaceutical composition comprising an immune cell that has been treated with, or which is in admixture with, an L-type Cav channel agonist.

15. The pharmaceutical composition of claim 14 for use in a method of stimulating cell killing activity against a target cell population.

16. The L-type Cav channel agonist for use of claim 13 or the pharmaceutical composition of claims 14 or 15, wherein the immune cell is selected from the group consisting of a T cell, a CAR-T cell, an NK cell and a CAR-NK cell.

17. An in vitro method of enhancing cell killing activity of an immune cell, comprising bringing an L-type Cav channel agonist into contact with the immune cell, wherein the L-type Cav channel agonist is capable of stimulating L-type Cav channel activity in a Gli1 reporter assay.

18. The method of claim 17, wherein the L-type Cav channel agonist is selected from the group consisting of FPL 64176 and Bay K8644.

19. An in vitro method of engineering an immune cell to increase cell killing activity, comprising genetically engineering an immune cell to enhance expression of or to stimulate L-type voltage-gated calcium (Cav) channel activity in a Gli1 reporter assay.

20. The method of claim 19, wherein said engineering comprises mutating Cav1.4 to introduce a gain-of-function mutation.

21. The method of claim 20, wherein the Cav1.4 mutation comprises I745T.

22. The method of any one of claims 19 to 21, wherein said engineering comprises overexpressing Cav1.4.

23. The method of any one of claims 19 to 22, wherein said engineering comprises nuclease or CRISPR-based editing of the CACNA1F gene.

24. The method of any one of claims 17 to 23, wherein the immune cell is selected from the group consisting of a T cell, a CAR-T cell, an NK cell and a CAR-NK cell.

25. A method of treatment of a proliferative disorder in a mammalian subject, comprising administering a therapeutically effective amount of an engineered immune cell to the subject in need thereof, wherein the immune cell comprises a gain-of-function mutation in an L-type Cav channel and/or overexpresses an L-type Cav channel.

26. The method of claim 25, wherein the immune cell is selected from the group consisting of a T cell, a CAR-T cell, an NK cell and a CAR-NK cell.

27. A method of treatment of a proliferative disorder in a mammalian subject, comprising administering a therapeutically effective amount of an L-type Cav channel agonist to the subject in need thereof.

28. A method of treatment of a proliferative disorder in a mammalian subject, comprising administering a therapeutically effective amount of an immune cell treated with an L-type Cav channel agonist to the subject in need thereof.

29. The method of claim 27 or 28, wherein the L-type Cav channel agonist is selected from the group consisting of FPL 64176 and Bay K8644.

30. An engineered immune cell for use in a method of treating chronic infection in a subject, wherein the engineered immune cell comprises a gain-of-function mutation in an L-type Cav channel and/or overexpresses an L-type Cav channel.

31. An engineered immune cell for use in a method of treating an autoimmune disease in a subject, wherein the engineered immune cell comprises a gain-of-function mutation in an L-type Cav channel and/or overexpresses an L-type Cav channel.

32. An in vitro method for determining the cytotoxic fitness of an immune cell from a mammalian subject, comprising measuring the level of GLI1 expression in the immune cell from the mammalian subject.

33. The method of claim 32, wherein the immune cell is an engineered immune cell.

34. The method of claim 32, wherein the method comprises administering a test agent to the immune cell.

35. The method of claim 34, wherein the test agent is an L-type Cav channel agonist.

36. The method of any one of claims 32 to 35, wherein the immune cell is a CAR T cell, a CAR NK cell, a T cell or an NK cell.

37. An engineered immune cell for use in a method of treating a proliferative disorder in a subject, wherein the engineered immune cell comprises a loss-of-function mutation in PTCH1 and/or wherein PTCH1 has been silenced or down-regulated and/or wherein the PTCH1 gene product has been antagonised.

38. The engineered immune cell for use according to claim 37, wherein the loss-of-function mutation in PTCH1 is a mutation that has been found to be present in a human patient having Gorlin Syndrome.

39. The engineered immune cell for use according to claim 37 or claim 38, wherein the immune cell is selected from the group consisting of a T cell, a CAR-T cell, an NK cell, and a CAR-NK cell.

40. A method of treatment of a proliferative disorder in a mammalian subject, comprising administering a therapeutically effective amount of an engineered immune cell to the subject in need thereof, wherein the immune cell is as defined in any one of claims 37 to 39.

41. The engineered immune cell for use according to any one of claims 37 to 39 or the method according to claim 40, wherein the proliferative disorder comprises a cancer, optionally wherein the cancer is selected from the group consisting of: melanoma, lung cancer, colorectal cancer, breast cancer, lymphoma, leukaemia, multiple myeloma, liver cancer, stomach cancer, central nervous system (CNS) tumour, paediatric glioma, ovarian cancer, cervical cancer, colon cancer, pancreatic cancer, prostate cancer, and gastric cancer.
